SEQUENCE LISTING (83 sequences)

SEQ ID NO: 1 - RNA, 21 nt, Artificial Sequence
  Description of Artificial Sequence Synthetic oligonucleotide
aagccccagccaguggggguu

SEQ ID NO: 2 - RNA, 19 nt, Artificial Sequence
  Description of Artificial Sequence Synthetic oligonucleotide
ccaagccccagccaguggg

SEQ ID NO: 3 - RNA, 20 nt, Artificial Sequence
  Description of Artificial Sequence Synthetic oligonucleotide
cuuguaaguuggcgagaaag

SEQ ID NO: 4 - RNA, 19 nt, Artificial Sequence
  Description of Artificial Sequence Synthetic oligonucleotide
uacuuuccaaucaauaggc

SEQ ID NO: 5 - RNA, 17 nt, Artificial Sequence
  Description of Artificial Sequence Synthetic oligonucleotide
ccuauugauuggaaagu

SEQ ID NO: 6 - RNA, 19 nt, Artificial Sequence
  Description of Artificial Sequence Synthetic oligonucleotide
gccuauugauuggaaagua

SEQ ID NO: 7 - RNA, 20 nt, Artificial Sequence
  Description of Artificial Sequence Synthetic oligonucleotide
ugaaccuuuaccccguugcc

SEQ ID NO: 8 - RNA, 20 nt, Artificial Sequence
  Description of Artificial Sequence Synthetic oligonucleotide
ugcgucagcaaacacuuggc

SEQ ID NO: 9 - RNA, 20 nt, Artificial Sequence
  Description of Artificial Sequence Synthetic oligonucleotide
gcgucagcaaacacuuggca

SEQ ID NO: 10 - RNA, 20 nt, Artificial Sequence
  Description of Artificial Sequence Synthetic oligonucleotide
ucucgccaacuuacaaggcc

SEQ ID NO: 11 - RNA, 14 nt, Artificial Sequence
  Description of Artificial Sequence Synthetic oligonucleotide
auugauuggaaagu

SEQ ID NO: 12 - RNA, 19 nt, Artificial Sequence
  Description of Artificial Sequence Synthetic oligonucleotide
gccaaguguuugcugacgc

SEQ ID NO: 13 - RNA, 19 nt, Artificial Sequence
  Description of Artificial Sequence Synthetic oligonucleotide
gcucgcagccggucuggag

SEQ ID NO: 14 - RNA, 14 nt, Artificial Sequence
  Description of Artificial Sequence Synthetic oligonucleotide
acuuuccaaucaau

SEQ ID NO: 15 - RNA, 20 nt, Artificial Sequence
  Description of Artificial Sequence Synthetic oligonucleotide
ggcaacgggguaaagguuca

SEQ ID NO: 16 - RNA, 20 nt, Artificial Sequence
  Description of Artificial Sequence Synthetic oligonucleotide
gccgggcaacgggguaaagg

SEQ ID NO: 17 - RNA, 19 nt, Artificial Sequence
  Description of Artificial Sequence Synthetic oligonucleotide
gcgucagcaaacacuuggc

SEQ ID NO: 18 - RNA, 20 nt, Artificial Sequence
  Description of Artificial Sequence Synthetic oligonucleotide
ccacgcaugcgcugauggcc

SEQ ID NO: 19 - RNA, 21 nt, Artificial Sequence
  Description of Artificial Sequence Synthetic oligonucleotide
ccagccaguggggguugcguc

SEQ ID NO: 20 - RNA, 20 nt, Artificial Sequence
  Description of Artificial Sequence Synthetic oligonucleotide
gcccccagccaguggggguu

SEQ ID NO: 21 - RNA, 19 nt, Artificial Sequence
  Description of Artificial Sequence Synthetic oligonucleotide
aagccccagccaguggggg

SEQ ID NO: 22 - RNA, 19 nt, Artificial Sequence
  Description of Artificial Sequence Synthetic oligonucleotide
agccaguggggguugcguc

SEQ ID NO: 23 - RNA, 20 nt, Artificial Sequence
  Description of Artificial Sequence Synthetic oligonucleotide
uuccacgcaugcgcugaugg

SEQ ID NO: 24 - RNA, 20 nt, Artificial Sequence
  Description of Artificial Sequence Synthetic oligonucleotide
aaagguuccacgcaugcgca

SEQ ID NO: 25 - RNA, 19 nt, Artificial Sequence
  Description of Artificial Sequence Synthetic oligonucleotide
uuccgcaguauggaucggc

SEQ ID NO: 26 - RNA, 21 nt, Artificial Sequence
  Description of Artificial Sequence Synthetic oligonucleotide
cgcaguauggaucggcagagg

SEQ ID NO: 27 - RNA, 21 nt, Artificial Sequence
  Description of Artificial Sequence Synthetic oligonucleotide
aggaguuccgcaguauggauc

SEQ ID NO: 28 - RNA, 19 nt, Artificial Sequence
  Description of Artificial Sequence Synthetic oligonucleotide
ggcugcgagcaaaacaagc

SEQ ID NO: 29 - RNA, 21 nt, Artificial Sequence
  Description of Artificial Sequence Synthetic oligonucleotide
ccggcugcgagcaaaacaagc

SEQ ID NO: 30 - RNA, 19 nt, Artificial Sequence
  Description of Artificial Sequence Synthetic oligonucleotide
ccggcugcgagcaaaacaa

SEQ ID NO: 31 - RNA, 21 nt, Artificial Sequence
  Description of Artificial Sequence Synthetic oligonucleotide
ccagaccggcugcgagcaaaa

SEQ ID NO: 32 - RNA, 19 nt, Artificial Sequence
  Description of Artificial Sequence Synthetic oligonucleotide
cuccagaccggcugcgagc

SEQ ID NO: 33 - RNA, 20 nt, Artificial Sequence
  Description of Artificial Sequence Synthetic oligonucleotide
gcuccagaccggcugcgagc

SEQ ID NO: 34 - RNA, 20 nt, Artificial Sequence
  Description of Artificial Sequence Synthetic oligonucleotide
ccggcugcgagcaaaacaag

SEQ ID NO: 35 - RNA, 20 nt, Artificial Sequence
  Description of Artificial Sequence Synthetic oligonucleotide
uuugcuccagaccggcugcg

SEQ ID NO: 36 - RNA, 20 nt, Artificial Sequence
  Description of Artificial Sequence Synthetic oligonucleotide
ugcuccagaccggcugcgag

SEQ ID NO: 37 - RNA, 20 nt, Artificial Sequence
  Description of Artificial Sequence Synthetic oligonucleotide
accggcugcgagcaaaacaa

SEQ ID NO: 38 - RNA, 20 nt, Artificial Sequence
  Description of Artificial Sequence Synthetic oligonucleotide
guugccgggcaacgggguaa

SEQ ID NO: 39 - RNA, 20 nt, Artificial Sequence
  Description of Artificial Sequence Synthetic oligonucleotide
uugccgggcaacgggguaaa

SEQ ID NO: 40 - RNA, 20 nt, Artificial Sequence
  Description of Artificial Sequence Synthetic oligonucleotide
gccgggcaacgggguaaagg

SEQ ID NO: 41 - RNA, 20 nt, Artificial Sequence
  Description of Artificial Sequence Synthetic oligonucleotide
ccgggcaacgggguaaaggu

SEQ ID NO: 42 - RNA, 20 nt, Artificial Sequence
  Description of Artificial Sequence Synthetic oligonucleotide
cgggcaacgggguaaagguu

SEQ ID NO: 43 - RNA, 20 nt, Artificial Sequence
  Description of Artificial Sequence Synthetic oligonucleotide
caguggggguugcgucagca

SEQ ID NO: 44 - RNA, 20 nt, Artificial Sequence
  Description of Artificial Sequence Synthetic oligonucleotide
aguggggguugcgucagcaa

SEQ ID NO: 45 - RNA, 20 nt, Artificial Sequence
  Description of Artificial Sequence Synthetic oligonucleotide
guggggguugcgucagcaaa

SEQ ID NO: 46 - RNA, 20 nt, Artificial Sequence
  Description of Artificial Sequence Synthetic oligonucleotide
uggggguugcgucagcaaac

SEQ ID NO: 47 - RNA, 20 nt, Artificial Sequence
  Description of Artificial Sequence Synthetic oligonucleotide
ggggguugcgucagcaaaca

SEQ ID NO: 48 - RNA, 20 nt, Artificial Sequence
  Description of Artificial Sequence Synthetic oligonucleotide
gggguugcgucagcaaacac

SEQ ID NO: 49 - RNA, 20 nt, Artificial Sequence
  Description of Artificial Sequence Synthetic oligonucleotide
gguugcgucagcaaacacuu

SEQ ID NO: 50 - RNA, 20 nt, Artificial Sequence
  Description of Artificial Sequence Synthetic oligonucleotide
ucgccaacuuacaaggccuu

SEQ ID NO: 51 - RNA, 20 nt, Artificial Sequence
  Description of Artificial Sequence Synthetic oligonucleotide
cucgccaacuuacaaggccu

SEQ ID NO: 52 - RNA, 20 nt, Artificial Sequence
  Description of Artificial Sequence Synthetic oligonucleotide
uucucgccaacuuacaaggc

SEQ ID NO: 53 - RNA, 20 nt, Artificial Sequence
  Description of Artificial Sequence Synthetic oligonucleotide
uuucucgccaacuuacaagg

SEQ ID NO: 54 - RNA, 20 nt, Artificial Sequence
  Description of Artificial Sequence Synthetic oligonucleotide
cuuucucgccaacuuacaag

SEQ ID NO: 55 - RNA, 20 nt, Artificial Sequence
  Description of Artificial Sequence Synthetic oligonucleotide
acuuucucgccaacuuacaa

SEQ ID NO: 56 - RNA, 19 nt, Artificial Sequence
  Description of Artificial Sequence Synthetic oligonucleotide
ugaaccuuuaccccguugc

SEQ ID NO: 57 - RNA, 20 nt, Artificial Sequence
  Description of Artificial Sequence Synthetic oligonucleotide
ccuuuaccccguugcccggc

SEQ ID NO: 58 - RNA, 21 nt, Artificial Sequence
  Description of Artificial Sequence Synthetic oligonucleotide
gauccauacugcggaacuccu

SEQ ID NO: 59 - RNA, 19 nt, Artificial Sequence
  Description of Artificial Sequence Synthetic oligonucleotide
gcuuguuuugcucgcagcc

SEQ ID NO: 60 - RNA, 21 nt, Artificial Sequence
  Description of Artificial Sequence Synthetic oligonucleotide
gcuuguuuugcucgcagccgg

SEQ ID NO: 61 - RNA, 19 nt, Artificial Sequence
  Description of Artificial Sequence Synthetic oligonucleotide
uuguuuugcucgcagccgg

SEQ ID NO: 62 - RNA, 19 nt, Artificial Sequence
  Description of Artificial Sequence Synthetic oligonucleotide
uuuugcucgcagccggucu

SEQ ID NO: 63 - RNA, 21 nt, Artificial Sequence
  Description of Artificial Sequence Synthetic oligonucleotide
uuuugcucgcagccggucugg

SEQ ID NO: 64 - RNA, 20 nt, Artificial Sequence
  Description of Artificial Sequence Synthetic oligonucleotide
gcucgcagccggucuggagc

SEQ ID NO: 65 - RNA, 20 nt, Artificial Sequence
  Description of Artificial Sequence Synthetic oligonucleotide
cuuguuuugcucgcagccgg

SEQ ID NO: 66 - RNA, 21 nt, Artificial Sequence
  Description of Artificial Sequence Synthetic oligonucleotide
  modified_base (1)..(1): 2′-O-Me A
  modified_base (2)..(2): 2′-O-Me A
  modified_base (3)..(3): 2′-O-Me G
  modified_base (4)..(4): 2′-O-Me, 5-methylcytosine
  modified_base (5)..(5): 2′-O-Me, 5-methylcytosine
  modified_base (6)..(6): 2′-O-Me, 5-methylcytosine
  modified_base (7)..(7): 2′-O-Me, 5-methylcytosine
  modified_base (8)..(8): 2′-O-Me A
  modified_base (9)..(9): 2′-O-Me G
  modified_base (10)..(10): 2′-O-Me, 5-methylcytosine
  modified_base (11)..(11): 2′-O-Me, 5-methylcytosine
  modified_base (12)..(12): 2′-O-Me A
  modified_base (13)..(13): 2′-O-Me G
  modified_base (14)..(14): 2′-O-Me U
  modified_base (15)..(15): 2′-O-Me G
  modified_base (16)..(16): 2′-O-Me G
  modified_base (17)..(17): 2′-O-Me G
  modified_base (18)..(18): 2′-O-Me G
  modified_base (19)..(19): 2′-O-Me G
  modified_base (20)..(20): 2′-O-Me U
  modified_base (21)..(21): 2′-O-Me U
  misc_feature (1)..(21): Phosphorothioate linkage
aagccccagccaguggggguu

SEQ ID NO: 67 - RNA, 19 nt, Artificial Sequence
  Description of Artificial Sequence Synthetic oligonucleotide
  modified_base (1)..(1): 2′-O-Me, 5-methylcytosine
  modified_base (2)..(2): 2′-O-Me, 5-methylcytosine
  modified_base (3)..(3): 2′-O-Me A
  modified_base (4)..(4): 2′-O-Me A
  modified_base (5)..(5): 2′-O-Me G
  modified_base (6)..(6): 2′-O-Me, 5-methylcytosine
  modified_base (7)..(7): 2′-O-Me, 5-methylcytosine
  modified_base (8)..(8): 2′-O-Me, 5-methylcytosine
  modified_base (9)..(9): 2′-O-Me, 5-methylcytosine
  modified_base (10)..(10): 2′-O-Me A
  modified_base (11)..(11): 2′-O-Me G
  modified_base (12)..(12): 2′-O-Me, 5-methylcytosine
  modified_base (13)..(13): 2′-O-Me, 5-methylcytosine
  modified_base (14)..(14): 2′-O-Me A
  modified_base (15)..(15): 2′-O-Me G
  modified_base (16)..(16): 2′-O-Me U
  modified_base (17)..(17): 2′-O-Me G
  modified_base (18)..(18): 2′-O-Me G
  modified_base (19)..(19): 2′-O-Me G
  misc_feature (1)..(19): Phosphorothioate linkage
ccaagccccagccaguggg

SEQ ID NO: 68 - RNA, 20 nt, Artificial Sequence
  Description of Artificial Sequence Synthetic oligonucleotide
  modified_base (1)..(1): 2′-O-Me, 5-methylcytosine
  modified_base (2)..(2): 2′-O-Me U
  modified_base (3)..(3): 2′-O-Me U
  modified_base (4)..(4): 2′-O-Me G
  modified_base (5)..(5): 2′-O-Me U
  modified_base (6)..(6): 2′-O-Me A
  modified_base (7)..(7): 2′-O-Me A
  modified_base (8)..(8): 2′-O-Me G
  modified_base (9)..(9): 2′-O-Me U
  modified_base (10)..(10): 2′-O-Me U
  modified_base (11)..(11): 2′-O-Me G
  modified_base (12)..(12): 2′-O-Me G
  modified_base (13)..(13): 2′-O-Me, 5-methylcytosine
  modified_base (14)..(14): 2′-O-Me G
  modified_base (15)..(15): 2′-O-Me A
  modified_base (16)..(16): 2′-O-Me G
  modified_base (17)..(17): 2′-O-Me A
  modified_base (18)..(18): 2′-O-Me A
  modified_base (19)..(19): 2′-O-Me A
  modified_base (20)..(20): 2′-O-Me G
  misc_feature (1)..(20): Phosphorothioate linkage
cuuguaaguuggcgagaaag

SEQ ID NO: 69 - RNA, 19 nt, Artificial Sequence
  Description of Artificial Sequence Synthetic oligonucleotide
  modified_base (1)..(1): 2′-O-Me U
  modified_base (2)..(2): 2′-O-Me A
  modified_base (3)..(3): 2′-O-Me, 5-methylcytosine
  modified_base (4)..(4): 2′-O-Me U
  modified_base (5)..(5): 2′-O-Me U
  modified_base (6)..(6): 2′-O-Me U
  modified_base (7)..(7): 2′-O-Me, 5-methylcytosine
  modified_base (8)..(8): 2′-O-Me, 5-methylcytosine
  modified_base (9)..(9): 2′-O-Me A
  modified_base (10)..(10): 2′-O-Me A
  modified_base (11)..(11): 2′-O-Me U
  modified_base (12)..(12): 2′-O-Me, 5-methylcytosine
  modified_base (13)..(13): 2′-O-Me A
  modified_base (14)..(14): 2′-O-Me A
  modified_base (15)..(15): 2′-O-Me U
  modified_base (16)..(16): 2′-O-Me A
  modified_base (17)..(17): 2′-O-Me G
  modified_base (18)..(18): 2′-O-Me G
  modified_base (19)..(19): 2′-O-Me, 5-methylcytosine
  misc_feature (1)..(19): Phosphorothioate linkage
uacuuuccaaucaauaggc

SEQ ID NO: 70 - RNA, 17 nt, Artificial Sequence
  Description of Artificial Sequence Synthetic oligonucleotide
  modified_base (1)..(1): 2′-O-Me, 5-methylcytosine
  modified_base (2)..(2): 2′-O-Me, 5-methylcytosine
  modified_base (3)..(3): 2′-O-Me U
  modified_base (4)..(4): 2′-O-Me A
  modified_base (5)..(5): 2′-O-Me U
  modified_base (6)..(6): 2′-O-Me U
  modified_base (7)..(7): 2′-O-Me G
  modified_base (8)..(8): 2′-O-Me A
  modified_base (9)..(9): 2′-O-Me U
  modified_base (10)..(10): 2′-O-Me U
  modified_base (11)..(11): 2′-O-Me G
  modified_base (12)..(12): 2′-O-Me G
  modified_base (13)..(13): 2′-O-Me A
  modified_base (14)..(14): 2′-O-Me A
  modified_base (15)..(15): 2′-O-Me A
  modified_base (16)..(16): 2′-O-Me G
  modified_base (17)..(17): 2′-O-Me U
  misc_feature (1)..(17): Phosphorothioate linkage
ccuauugauuggaaagu

SEQ ID NO: 71 - RNA, 19 nt, Artificial Sequence
  Description of Artificial Sequence Synthetic oligonucleotide
  modified_base (1)..(1): 2′-O-Me G
  modified_base (2)..(2): 2′-O-Me, 5-methylcytosine
  modified_base (3)..(3): 2′-O-Me, 5-methylcytosine
  modified_base (4)..(4): 2′-O-Me U
  modified_base (5)..(5): 2′-O-Me A
  modified_base (6)..(6): 2′-O-Me U
  modified_base (7)..(7): 2′-O-Me U
  modified_base (8)..(8): 2′-O-Me G
  modified_base (9)..(9): 2′-O-Me A
  modified_base (10)..(10): 2′-O-Me U
  modified_base (11)..(11): 2′-O-Me U
  modified_base (12)..(12): 2′-O-Me G
  modified_base (13)..(13): 2′-O-Me G
  modified_base (14)..(14): 2′-O-Me A
  modified_base (15)..(15): 2′-O-Me A
  modified_base (16)..(16): 2′-O-Me A
  modified_base (17)..(17): 2′-O-Me G
  modified_base (18)..(18): 2′-O-Me U
  modified_base (19)..(19): 2′-O-Me A
  misc_feature (1)..(19): Phosphorothioate linkage
gccuauugauuggaaagua

SEQ ID NO: 72 - RNA, 20 nt, Artificial Sequence
  Description of Artificial Sequence Synthetic oligonucleotide
  modified_base (1)..(1): 2′-O-Me U
  modified_base (2)..(2): 2′-O-Me G
  modified_base (3)..(3): 2′-O-Me A
  modified_base (4)..(4): 2′-O-Me A
  modified_base (5)..(5): 2′-O-Me, 5-methylcytosine
  modified_base (6)..(6): 2′-O-Me, 5-methylcytosine
  modified_base (7)..(7): 2′-O-Me U
  modified_base (8)..(8): 2′-O-Me U
  modified_base (9)..(9): 2′-O-Me U
  modified_base (10)..(10): 2′-O-Me A
  modified_base (11)..(11): 2′-O-Me, 5-methylcytosine
  modified_base (12)..(12): 2′-O-Me, 5-methylcytosine
  modified_base (13)..(13): 2′-O-Me, 5-methylcytosine
  modified_base (14)..(14): 2′-O-Me, 5-methylcytosine
  modified_base (15)..(15): 2′-O-Me G
  modified_base (16)..(16): 2′-O-Me U
  modified_base (17)..(17): 2′-O-Me U
  modified_base (18)..(18): 2′-O-Me G
  modified_base (19)..(19): 2′-O-Me, 5-methylcytosine
  modified_base (20)..(20): 2′-O-Me, 5-methylcytosine
  misc_feature (1)..(20): Phosphorothioate linkage
ugaaccuuuaccccguugcc

SEQ ID NO: 73 - RNA, 20 nt, Artificial Sequence
  Description of Artificial Sequence Synthetic oligonucleotide
  modified_base (1)..(1): 2′-O-Me U
  modified_base (2)..(2): 2′-O-Me G
  modified_base (3)..(3): 2′-O-Me A
  modified_base (4)..(4): 2′-O-Me A
  modified_base (5)..(5): 2′-O-Me, 5-methylcytosine
  modified_base (6)..(6): 2′-O-Me, 5-methylcytosine
  modified_base (7)..(7): 2′-O-Me U
  modified_base (8)..(8): 2′-O-Me U
  modified_base (9)..(9): 2′-O-Me U
  modified_base (10)..(10): 2′-O-Me A
  modified_base (11)..(11): 2′-O-Me, 5-methylcytosine
  modified_base (12)..(12): 2′-O-Me, 5-methylcytosine
  modified_base (13)..(13): 2′-O-Me, 5-methylcytosine
  modified_base (14)..(14): 2′-O-Me, 5-methylcytosine
  modified_base (15)..(15): 2′-O-Me G
  modified_base (16)..(16): 2′-O-Me U
  modified_base (17)..(17): 2′-O-Me U
  modified_base (18)..(18): 2′-O-Me G
  modified_base (19)..(19): 2′-O-Me, 5-methylcytosine
  modified_base (20)..(20): 2′-O-Me, 5-methylcytosine
  misc_feature (1)..(20): Thiophosphoramidate linkage
ugaaccuuuaccccguugcc

SEQ ID NO: 74 - RNA, 20 nt, Artificial Sequence
  Description of Artificial Sequence Synthetic oligonucleotide
  modified_base (1)..(1): 2′-O-Me U
  modified_base (2)..(2): 2′-O-Me G
  modified_base (3)..(3): 2′-O-Me, 5-methylcytosine
  modified_base (4)..(4): 2′-O-Me G
  modified_base (5)..(5): 2′-O-Me U
  modified_base (6)..(6): 2′-O-Me, 5-methylcytosine
  modified_base (7)..(7): 2′-O-Me A
  modified_base (8)..(8): 2′-O-Me G
  modified_base (9)..(9): 2′-O-Me, 5-methylcytosine
  modified_base (10)..(10): 2′-O-Me A
  modified_base (11)..(11): 2′-O-Me A
  modified_base (12)..(12): 2′-O-Me A
  modified_base (13)..(13): 2′-O-Me, 5-methylcytosine
  modified_base (14)..(14): 2′-O-Me A
  modified_base (15)..(15): 2′-O-Me, 5-methylcytosine
  modified_base (16)..(16): 2′-O-Me U
  modified_base (17)..(17): 2′-O-Me U
  modified_base (18)..(18): 2′-O-Me G
  modified_base (19)..(19): 2′-O-Me G
  modified_base (20)..(20): 2′-O-Me, 5-methylcytosine
  misc_feature (1)..(20): Phosphorothioate linkage
ugcgucagcaaacacuuggc

SEQ ID NO: 75 - RNA, 20 nt, Artificial Sequence
  Description of Artificial Sequence Synthetic oligonucleotide
  modified_base (1)..(1): 2′-O-Me G
  modified_base (2)..(2): 2′-O-Me, 5-methylcytosine
  modified_base (3)..(3): 2′-O-Me G
  modified_base (4)..(4): 2′-O-Me U
  modified_base (5)..(5): 2′-O-Me, 5-methylcytosine
  modified_base (6)..(6): 2′-O-Me A
  modified_base (7)..(7): 2′-O-Me G
  modified_base (8)..(8): 2′-O-Me, 5-methylcytosine
  modified_base (9)..(9): 2′-O-Me A
  modified_base (10)..(10): 2′-O-Me A
  modified_base (11)..(11): 2′-O-Me A
  modified_base (12)..(12): 2′-O-Me, 5-methylcytosine
  modified_base (13)..(13): 2′-O-Me A
  modified_base (14)..(14): 2′-O-Me, 5-methylcytosine
  modified_base (15)..(15): 2′-O-Me U
  modified_base (16)..(16): 2′-O-Me U
  modified_base (17)..(17): 2′-O-Me G
  modified_base (18)..(18): 2′-O-Me G
  modified_base (19)..(19): 2′-O-Me, 5-methylcytosine
  modified_base (20)..(20): 2′-O-Me A
  misc_feature (1)..(20): Phosphorothioate linkage
gcgucagcaaacacuuggca

SEQ ID NO: 76 - RNA, 20 nt, Artificial Sequence
  Description of Artificial Sequence Synthetic oligonucleotide
  modified_base (1)..(1): 2′-O-Me U
  modified_base (2)..(2): 2′-O-Me, 5-methylcytosine
  modified_base (3)..(3): 2′-O-Me U
  modified_base (4)..(4): 2′-O-Me, 5-methylcytosine
  modified_base (5)..(5): 2′-O-Me G
  modified_base (6)..(6): 2′-O-Me, 5-methylcytosine
  modified_base (7)..(7): 2′-O-Me, 5-methylcytosine
  modified_base (8)..(8): 2′-O-Me A
  modified_base (9)..(9): 2′-O-Me A
  modified_base (10)..(10): 2′-O-Me, 5-methylcytosine
  modified_base (11)..(11): 2′-O-Me U
  modified_base (12)..(12): 2′-O-Me U
  modified_base (13)..(13): 2′-O-Me A
  modified_base (14)..(14): 2′-O-Me, 5-methylcytosine
  modified_base (15)..(15): 2′-O-Me A
  modified_base (16)..(16): 2′-O-Me A
  modified_base (17)..(17): 2′-O-Me G
  modified_base (18)..(18): 2′-O-Me G
  modified_base (19)..(19): 2′-O-Me, 5-methylcytosine
  modified_base (20)..(20): 2′-O-Me, 5-methylcytosine
  misc_feature (1)..(20): Phosphorothioate linkage
ucucgccaacuuacaaggcc

SEQ ID NO: 77 - RNA, 14 nt, Artificial Sequence
  Description of Artificial Sequence Synthetic oligonucleotide
  modified_base (1)..(1): 2′-O-Me A
  modified_base (2)..(2): 2′-O-Me U
  modified_base (3)..(3): 2′-O-Me U
  modified_base (4)..(4): 2′-O-Me G
  modified_base (5)..(5): 2′-O-Me A
  modified_base (6)..(6): 2′-O-Me U
  modified_base (7)..(7): 2′-O-Me U
  modified_base (8)..(8): 2′-O-Me G
  modified_base (9)..(9): 2′-O-Me G
  modified_base (10)..(10): 2′-O-Me A
  modified_base (11)..(11): 2′-O-Me A
  modified_base (12)..(12): 2′-O-Me A
  modified_base (13)..(13): 2′-O-Me G
  modified_base (14)..(14): 2′-O-Me U
  misc_feature (1)..(14): Phosphorothioate linkage
auugauuggaaagu

SEQ ID NO: 78 - RNA, 19 nt, Artificial Sequence
  Description of Artificial Sequence Synthetic oligonucleotide
  modified_base (1)..(1): 2′-O-Me G
  modified_base (2)..(2): 2′-O-Me, 5-methylcytosine
  modified_base (3)..(3): 2′-O-Me, 5-methylcytosine
  modified_base (4)..(4): 2′-O-Me A
  modified_base (5)..(5): 2′-O-Me A
  modified_base (6)..(6): 2′-O-Me G
  modified_base (7)..(7): 2′-O-Me U
  modified_base (8)..(8): 2′-O-Me G
  modified_base (9)..(9): 2′-O-Me U
  modified_base (10)..(10): 2′-O-Me U
  modified_base (11)..(11): 2′-O-Me U
  modified_base (12)..(12): 2′-O-Me G
  modified_base (13)..(13): 2′-O-Me, 5-methylcytosine
  modified_base (14)..(14): 2′-O-Me U
  modified_base (15)..(15): 2′-O-Me G
  modified_base (16)..(16): 2′-O-Me A
  modified_base (17)..(17): 2′-O-Me, 5-methylcytosine
  modified_base (18)..(18): 2′-O-Me G
  modified_base (19)..(19): 2′-O-Me, 5-methylcytosine
  misc_feature (1)..(19): Phosphorothioate linkage
gccaaguguuugcugacgc

SEQ ID NO: 79 - RNA, 19 nt, Artificial Sequence
  Description of Artificial Sequence Synthetic oligonucleotide
  modified_base (1)..(1): 2′-O-Me G
  modified_base (2)..(2): 2′-O-Me, 5-methylcytosine
  modified_base (3)..(3): 2′-O-Me U
  modified_base (4)..(4): 2′-O-Me, 5-methylcytosine
  modified_base (5)..(5): 2′-O-Me G
  modified_base (6)..(6): 2′-O-Me, 5-methylcytosine
  modified_base (7)..(7): 2′-O-Me A
  modified_base (8)..(8): 2′-O-Me G
  modified_base (9)..(9): 2′-O-Me, 5-methylcytosine
  modified_base (10)..(10): 2′-O-Me, 5-methylcytosine
  modified_base (11)..(11): 2′-O-Me G
  modified_base (12)..(12): 2′-O-Me G
  modified_base (13)..(13): 2′-O-Me U
  modified_base (14)..(14): 2′-O-Me, 5-methylcytosine
  modified_base (15)..(15): 2′-O-Me U
  modified_base (16)..(16): 2′-O-Me G
  modified_base (17)..(17): 2′-O-Me G
  modified_base (18)..(18): 2′-O-Me A
  modified_base (19)..(19): 2′-O-Me G
  misc_feature (1)..(19): Phosphorothioate linkage
gcucgcagccggucuggag

SEQ ID NO: 80 - RNA, 19 nt, Artificial Sequence
  Description of Artificial Sequence Synthetic oligonucleotide
  3′-GalNAc
gccuauugauuggaaagua

SEQ ID NO: 81 - RNA, 19 nt, Artificial Sequence
  Description of Artificial Sequence Synthetic oligonucleotide
  3′-palmitoyl
gccuauugauuggaaagua

SEQ ID NO: 82 - RNA, 19 nt, Artificial Sequence
  Description of Artificial Sequence Synthetic oligonucleotide
  3′ tocopherol
gccuauugauuggaaagua

SEQ ID NO: 83 - DNA, 3213 bp, Artificial Sequence
  Description of Artificial Sequence Synthetic polynucleotide
ctccacaacattccaccaagctctgcaagatcccagagtgaggggcctgtattttcctgctggtggctccagttcaggaacagtaaaccctgttccgactactgcctctcccatatcgtcaatcttctcgaggactggggaccctgcaccgaacatggagaacatcacatcaggattcctaggacccctgctcgtgttacaggcggggtttttcttgttgacaagaatcctcacaataccacagagtctagactcgtggtggacttctctcaattttctagggggaacacccgtgtgtcttggccaaaattcgcagtccccaacctccaatcactcaccaacctcttgtcctccaatttgtcctggttatcgctggatgtgtctgcggcgttttatcatcttcctcttcatcctgctgctatgcctcatcttcttgttggttcttctggactatcaaggtatgttgcccgtttgtcctctaattccaggatcatcaaccaccagcacgggaccatgcaaaacctgcacgactcctgctcaaggaacctctatgtttccctcatgttgctgtacaaaaccttcggacggaaactgcacctgtattcccatcccatcatcctgggctttcgcaaaattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttaccgctgttaccaattttcttttgtctttgggtatacatttaaaccctaacaaaacaaaaagatggggttactcccttaacttcatgggatatgtaattggaagttggggtacattgccacaggaacatattgtacaaaaaatcaaacaatgttttagaaaacttcctgtaaacaggcctattgattggaaagtatgtcaacgaattgtgggtcttttgggctttgctgccccttttacacaatgtggttatcctgctttaatgcctttatatgcatgtatacaagctaagcaggctttcactttctcgccaacttacaaggcctttctgtgtaaacaatatctgaacctttaccccgttgcccggcaacggccaggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggagcaaaacttatcgggactgacaactctgttgtcctctcccggaaatatacatcctttccatggctgctaggctgtgctgccaactggatcctgcgcgggacgtcctttgtttacgtcccgtcggcgctgaatcccgcggacgacccctctcggggccgcttgggactctaccgtccccttctccgtctgccgttccggccgaccacggggcgcacctctctttacgcggtctccccgtctgtgccttctcatctgccggaccgtgtgcacttcgcttcacctctgcacgtcgcatggagaccaccgtgaacgcccaccagcttgcccaaggtcttacataagaggactcttggactctcagcaatgtcaacgaccgaccttgaggcatacttcaaagactgtgtgtttaaagactgggaggagttgggggaggagattaggttaaaggtctttgtactaggaggctgtaggcataaattggtctgttcaccagcaccatgcaactttttcacctctgcctaatcatctcttgttcatgtcctactgttcaagcctccaagctgtgccttgggtggctttggggcatggacattgacccttataaagaatttggagcttctgtggagttactctcttttttgccttctgacttctttccttctattcgagatctcctcgacaccgcctcagctctgtatcgggaggccttagagtctccggaacattgttcacctcaccatacagcactcaggcaagctattctgtgttggggtgagttgatgaatctagccacctgggtgggaagtaatttggaagatccagcatccagggaattagtagtcagctatgtcaatgttaatatgggcctaaaaatcagacaactattgtggtttcacatttcctgtcttacttttggaagagaaactgttcttgagtatttggtgtcttttggagtgtggattcgcactcctccagcttatagaccaccaaatgcccctatcttatcaacacttccggaaactactgttgttagacgacgaggcaggtcccctagaagaagaactccctcgcctcgcagacgaaggtctcaatcgccgcgtcgcagaagatctcaatctcgggaatctcaatgttagtattccttggactcataaggtgggaaactttactgggctttattcttctactgtacctgtctttaatcctgaatggcaaactccctcttttcctaacattcatttacaggaggacattattaatagatgtcaacaatttgtgggccctcttacagtaaatgaaaaaaggagattaaaattaattatgcctgctaggttttatcctaaccttaccaaatatttgcccttagataaaggcattaaaccttattatccagaacatgtagttaatcattacttccaaaccagacattatttacatactctttggaaggcgggtattttatataagagagaaactacacgtagcgcctcattttgtgggtcaccatattcttgggaacaagagctacagcatgggaggttggtcttccaaacctcgaaaaggcatggggacgaatctttctgttcccaatcctctgggattctttcccgatcaccagttggaccctgcattcagagccaactcaaacaatccagattgggacttcaaccccaacaaggaccactggccagacgccaaccaggtaggagtgggagcattcgggccagggttcaccccaccacacggcggtcttttggggtggagccctcaggctcagggcatattgacaacagtgccagcagctcctcctcctgcctccaccaatcggcagtcaggaaggcagcctactcccatctctccacctctaagagacagtcatcctcaggccatgcagtggaa

(19) United States
(12) Patent Application Publication
(10) Pub. No.: US 2018/0179542 A1
(43) Pub. Date: Jun. 28, 2018

(54) OLIGONUCLEOTIDE TARGETING STRATEGY FOR cccDNA

(71) Applicant: Alios Biopharma, Inc., South San Francisco, CA (US)

(72) Inventors: Sergei Gryaznov, San Mateo, CA (US); Megan Fitzgerald, South San Francisco, CA (US); Antitsa Dimitrova Stoycheva, Half Moon Bay, CA (US); Jin Hong, Pacifica, CA (US)

(73) Assignee: Alios Biopharma, Inc., South San Francisco, CA (US)

(21) Appl. No.: 15/810,857

(22) Filed: Nov. 13, 2017

Related U.S. Application Data
(60) Provisional application No. 62/420,801, filed on Nov. 11, 2016.
(60) Provisional application No. 62/558,770, filed on Sep. 14, 2017.

(51) Int. Cl. C12N 15/113; A61P 31/20; A61K 31/7125; A61K 45/06

(52) U.S. Cl. CPC C12N 15/1131; A61P 31/20; A61K 31/7125; A61K 45/06; C12N 2310/314; C12N 2310/315; C12N 2310/34; C12N 2310/321; C12N 2310/3341; C12N 2310/11

(57) ABSTRACT

The present disclosure provides oligonucleotide compositions that target the covalently closed circular (ccc) DNA of hepatitis B virus (HBV). Also disclosed herein are methods for treating a subject diagnosed with, or suspected of having an HBV infection and/or an HBV-associated disorder, e.g., chronic hepatitis B infection, liver failure or cirrhosis and hepatocellular carcinoma.

### CROSS-REFERENCE TO RELATED APPLICATIONS

This application claims the benefit of and priority to U.S. Application Nos. 62/420,801 filed Nov. 11, 2016, and 62/558,770, filed Sep. 14, 2017, the contents of which are incorporated herein by reference in their entireties.

### TECHNICAL FIELD

The present disclosure relates to oligonucleotide compositions that target the covalently closed circular (ccc) DNA of hepatitis B virus (HBV) and methods of using the same to treat subjects diagnosed with, or suspected of having an HBV infection and/or an HBV-associated disorder, e.g., chronic hepatitis B infection.

### BACKGROUND

The following description of the background of the present disclosure is provided simply as an aid in understanding the present disclosure and is not intended as an admission that such disclosure is prior art to the present disclosure.

HBV is one of the few DNA viruses that utilize reverse transcriptase in the replication process which involves multiple stages including entry, uncoating and transport of the viral genome to the nucleus. Initially, replication of the HBV genome involves the generation of an RNA intermediate that is then reverse transcribed to produce the DNA viral genome. Upon infection of a cell with HBV, the viral genomic relaxed circular DNA (rcDNA) is transported into the cell nucleus and converted into episomal double-stranded covalently closed circular DNA (cccDNA), which serves as the transcription template for the viral mRNAs. After transcription and nuclear export, cytoplasmic viral pregenomic RNA (pgRNA) is assembled with HBV polymerase and capsid proteins to form the nucleocapsid, inside which polymerase-catalyzed reverse transcription yields minus-strand DNA, which is subsequently copied into plus-strand DNA to form the progeny rcDNA genome. The mature nucleocapsids are then either packaged with viral envelope proteins to egress as virion particles or shuttled to the nucleus to amplify the cccDNA reservoir through the intracellular cccDNA amplification pathway. cccDNA is an essential component of the HBV replication cycle and is responsible for the establishment of infection and viral persistence.

At least 250 million people worldwide are chronically infected with hepatitis B virus (HBV), a hepatotropic DNA virus that replicates through reverse transcription. Chronic infection greatly increases the risk for terminal liver disease. Current therapies rarely achieve a complete cure due to the refractory nature of an intracellular viral replication intermediate termed covalently closed circular (ccc) DNA. Upon infection, cccDNA is generated as a plasmid-like episome in the host cell nucleus from the protein-linked relaxed circular (RC) DNA genome in incoming virions. cccDNA serves as the template for all viral RNAs, and thus new virions. cccDNA can persist in patients recovering from acute HBV infection for decades.

Accumulating evidence demonstrates that current antiviral treatments alone, such as nucleos(t)ide analogs (NAs) or interferon (IFN), fail to cure most chronic hepatitis B (CHB) patients because of the persistent nature of cccDNA. NA suppresses HBV replication by directly inhibiting viral polymerase, while IFN enhances host immunity against HBV infection. Viral rebound often occurs after discontinuation of antiviral treatment.

There is therefore a need for novel antiviral agents targeting cccDNA or cccDNA-containing hepatocytes are thus required for curing chronic HBV infection.

### SUMMARY

The present disclosure is directed to an oligonucleotide and pharmaceutical compositions thereof. The oligonucleotide comprises a sequence that is complementary to a plurality of nucleotides within an HBV cccDNA genome sequence of SEQ ID NO: 100. In embodiments, the oligonucleotide comprises a sequence that is complementary to at least 12 nucleotides within the HBV cccDNA genome. In embodiments, the oligonucleotide is complementary to at least 12 nucleotides within the Enhancer I region of the HBV cccDNA genome. In embodiments, the oligonucleotide comprises a sequence that is complementary to at least 12 nucleotides that are present in a region corresponding to nucleotide position 967 to nucleotide position 1322 of the HBV cccDNA genome. In embodiments, the sequence of the oligonucleotide is any one of SEQ ID NOs: 1-65.

In embodiments, the disclosed oligonucleotides contain at least one first nucleotide having a phosphorothioate (PS) linkage or a thiophosphoramidate (NPS) linkage to a second nucleotide. In embodiments, the first nucleotide is further modified at the 2′ position with a substitution that includes a fluorine (F) or an O-alkyl such as O-methyl (O-Me), O-ethyl (O-Et) and the like. The O-alkyl may be further substituted with alkoxy such as O-methyl (O-Me), O-ethyl (O-Et) and the like. In embodiments, any first nucleotide having a cytosine nucleobase is further modified to be a methylcytosine.

In embodiments, each nucleotide of the disclosed oligonucleotides contains a phosphorothioate (PS) linkage or a thiophosphoramidate (NPS) linkage between the nucleotides along with an O-methyl substitution at the 2′ position, and any nucleotide having a cytosine nucleobase is further modified to include a methylcytosine nucleobase. In embodiments, each nucleotide of oligonucleotides having SEQ ID NOs: 1-65 are modified to contain a phosphorothioate (PS) linkage or a thiophosphoramidate (NPS) linkage between the nucleotides along with an O-methyl substitution at the 2′ position, and any nucleotide having a cytosine nucleobase is further modified to include a methylcytosine nucleobase. In embodiments, the sequence of the oligonucleotide is any one of SEQ ID NOs: 66-79.

In embodiments, the disclosed oligonucleotides are modified to contain at least one targeting moiety conjugated to the oligonucleotide. The targeting moiety conjugated to the oligonucleotide may be a GalNAc, palmitoyl or tocopherol derivative. In embodiments, oligonucleotides having SEQ ID Nos: 1-79 are modified to contain at least one targeting moiety conjugated to the oligonucleotide. In embodiments, the sequence of the oligonucleotide is any one of SEQ ID NOs: 80-82.

In embodiments, the pharmaceutical compositions comprise at least one oligonucleotide having a sequence that is complementary to at least 12 nucleotides within the HBV cccDNA genome. In embodiments, the oligonucleotide is complementary to at least 12 nucleotides within the Enhancer I region of the HBV cccDNA genome. In embodiments, the oligonucleotide comprises a sequence that is complementary to at least 12 nucleotides that are present in a region corresponding to nucleotide position 967 to nucleotide position 1322 of the HBV cccDNA genome. In embodiments, at least one first nucleotide of the oligonucleotide is modified to contain a phosphorothioate (PS) linkage or a thiophosphoramidate (NPS) linkage to a second nucleotide, and a fluorine (F) or an O-alkyl (optionally further substituted with alkoxy) substitution at the 2′ position, and any cytosine nucleobase is further modified to be a methylcytosine. In embodiments, the oligonucleotide is modified with a targeting moiety such as a GalNAc, palmitoyl or tocopherol derivative conjugated at the 3′ and/or 5′ end of the oligonucleotide. In embodiments, the sequence of the oligonucleotide in the pharmaceutical composition is any one of SEQ ID NOs: 1-82.

The present disclosure is further directed to methods of treating hepatitis B virus (HBV) infection in a subject in need thereof. The methods comprise administering to the subject an effective amount of the oligonucleotide of the present disclosure or pharmaceutical compositions thereof. In embodiments, the sequence of the oligonucleotide is any one of SEQ ID NOs: 1-82. In embodiments, administration of the oligonucleotide results in a decrease in at least one of HBeAg levels, HBsAg levels or HBV DNA levels in the subject. In embodiments, administration of the oligonucleotide results in a reduction of HBV cccDNA in the subject.

The methods of the present disclosure further comprise separately, sequentially or simultaneously administering to the subject one or more additional therapeutic agents selected from the group consisting of: an antiviral agent, a nucleotide analog, a nucleoside analog, a reverse transcriptase inhibitor, an immune modulator, a therapeutic vaccine, a viral entry inhibitor, a capsid inhibitor, a siRNA, an antisense oligonucleotide, and a cccDNA inhibitor.

In addition, the present disclosure is directed to the disclosed oligonucleotides for use in the treatment of HBV.

In one aspect, the present disclosure provides an oligonucleotide comprising a sequence selected from the group consisting of SEQ ID NOs: 1-82, or modifications thereof. The present disclosure also provides an oligonucleotide comprising a complementary sequence of any of SEQ ID NOs: 1-82, or modifications thereof.

In some embodiments, the oligonucleotides of the present disclosure target an HBV DNA sequence that is within the Enhancer I region of the HBV cccDNA genome (i.e., a target nucleotide sequence located between and including nucleotide positions 960 and 1330 of the HBV genome).

In some embodiments, the oligonucleotides of the present disclosure target an HBV DNA sequence that is located anywhere between position 969 and position 987 of the HBV genome. In certain embodiments, the oligonucleotides of the present disclosure target an HBV DNA sequence that is located anywhere between position 1094 and position 1116 of the HBV genome. In some embodiments, the oligonucleotides of the present disclosure target an HBV DNA sequence that is located anywhere between position 1136 and position 1155 of the HBV genome. In some embodiments, the oligonucleotides of the present disclosure target an HBV DNA sequence that is located anywhere between position 1174 and position 1194 of the HBV genome. In other embodiments, the oligonucleotides of the present disclosure target an HBV DNA sequence that is located anywhere between position 1194 and position 1216 of the HBV genome. In some embodiments, the oligonucleotides of the present disclosure target an HBV DNA sequence that is located anywhere between position 1297 and position 1315 of the HBV genome.

In one aspect, the present disclosure provides methods for treating an HBV infection or an HBV-associated disorder, and/or treating the signs or symptoms of an HBV infection or an HBV-associated disorder in a subject in need thereof, comprising administering to the subject a therapeutically effective amount of at least one oligonucleotide, wherein the at least one oligonucleotide comprises a sequence selected from the group consisting of SEQ ID NOs: 1-82.

In one aspect, the present disclosure provides a method for inducing D-loop formation in HBV cccDNA comprising contacting HBV cccDNA with an oligonucleotide having a sequence of any one of SEQ ID NOs: 1-82. In another aspect, the present disclosure provides a method for inducing D-loop formation in HBV cccDNA comprising contacting a target region of an HBV cccDNA genome consisting of nucleotide position 900-1310 (Enhancer I region) with an oligonucleotide that is at least 90% complementary to the target region of the HBV cccDNA. In some embodiments, the oligonucleotides disclosed herein hybridize with HBV cccDNA to induce the formation of an antigenic D-loop structure. In some embodiments, induction of D-loop formation stimulates innate immunity.

In one aspect, the present disclosure provides an oligonucleotide comprising a sequence that is complementary to a plurality of nucleotides near or within Enhancer I of an HBV cccDNA genome, wherein Enhancer I corresponds to nucleotide position 900 to nucleotide position 1310 of the HBV cccDNA genome sequence of SEQ ID NO: 100. In some embodiments, the oligonucleotide is complementary to at least 15 nucleotides within the Enhancer I region of the HBV cccDNA genome. In certain embodiments, the oligonucleotide is complementary to at least 19 nucleotides within the Enhancer I region of the HBV cccDNA genome.

In another aspect, the present disclosure provides an oligonucleotide comprising a sequence that is complementary to at least 15 nucleotides that are present in a genome region corresponding to nucleotide position 960 to nucleotide position 1330 of an HBV cccDNA genome, wherein the HBV cccDNA genome sequence is SEQ ID NO: 100. In some embodiments, the oligonucleotide comprises a sequence that is complementary to at least 19 nucleotides that are present in the genome region corresponding to nucleotide position 960 to nucleotide position 1330 of the HBV cccDNA genome. Additionally or alternatively, in some embodiments, the sequence of the oligonucleotide is selected from the group consisting of SEQ ID NOs: 1-82. Additionally or alternatively, in any of the above embodiments, the oligonucleotide contains at least one first nucleotide having a PS linkage to a second nucleotide, wherein said first nucleotide is modified at the 2′ position with a substitution selected from the group consisting of F and O-alkyl, wherein said O-alkyl is optionally substituted with alkoxy. Additionally or alternatively, in any of the above embodiments, each nucleotide of the oligonucleotide is linked to the other nucleotide of the oligonucleotide by a PS linkages and modified at the 2′ position with O-Me.

In another aspect, the present disclosure provides a method of treating HBV in a subject in need thereof comprising administering to the subject an effective amount of any of the oligonucleotides disclosed herein. In some embodiments, the oligonucleotide is complementary to at least 15 nucleotides within the Enhancer I region of the HBV cccDNA genome. In certain embodiments, the oligonucleotide comprises a sequence that is complementary to at least 19 nucleotides that are present in the genome region corresponding to nucleotide position 960 to nucleotide position 1330 of the HBV cccDNA genome. Additionally or alternatively, in some embodiments, the sequence of the oligonucleotide is selected from the group consisting of SEQ ID NOs: 1-82.

In certain embodiments, administration of the oligonucleotide results in a decrease in at least one of HBeAg levels, HBsAg levels or HBV DNA levels in the subject. Additionally or alternatively, in some embodiments, administration of the oligonucleotide results in a reduction of liver levels of HBV cccDNA in the subject. The oligonucleotide may be administered orally, topically, systemically, intravenously, subcutaneously, transdermally, intrathecally, intranasally, intraperitoneally, intrahepatically, or intramuscularly.

Additionally or alternatively, in some embodiments, the method further comprises separately, sequentially or simultaneously administering to the subject one or more additional therapeutic agents selected from the group consisting of: an antiviral agent, a nucleotide analog, a nucleoside analog, a reverse transcriptase inhibitor, an immune stimulator, a therapeutic vaccine, a viral entry inhibitor, a capsid inhibitor, a siRNA, an antisense oligonucleotide, and a cccDNA inhibitor.

In another aspect, the present disclosure provides an oligonucleotide for use in the treatment of HBV, wherein the oligonucleotide comprises a sequence that is complementary to a plurality of nucleotides near or within Enhancer I of an HBV cccDNA genome, wherein Enhancer I corresponds to nucleotide position 900 to nucleotide position 1310 of the HBV cccDNA genome of SEQ ID NO: 100. In some embodiments, the oligonucleotide comprises a sequence that is complementary to at least 15 nucleotides that are present in a genome region corresponding to nucleotide position 960 to nucleotide position 1330 of the HBV cccDNA genome. In certain embodiments, the sequence of the oligonucleotide is selected from the group consisting of SEQ ID NOs: 1-82.

### BRIEF DESCRIPTION OF THE DRAWINGS

FIG. 1 shows the physical map of the HBV genome. The HBV genome is approximately 3200 nucleotides in length, wherein the nucleotide sequence of the Enhancer I region starts at position 900 and ends at position 1310.

FIG. 2A shows the Southern Blot results of HBV infected PHH treated with various concentrations of SEQ ID NO: 71. FIG. 2B shows the qPCR results of HBV infected PHH treated with various concentrations of SEQ ID NO: 71. FIG. 2C shows the % reduction of cccDNA levels in HBV infected PHH treated with various concentrations of SEQ ID NO: 71. FIG. 2D shows the Southern Blot results of HBV infected PHH treated with various concentrations of SEQ ID NO: 70, SEQ ID NO: 72 and SEQ ID NO: 75. In FIGS. 2A-2D, PHH were infected with HBV at day 0, and treated with the indicated cccDNA targeting oligonucleotide at day 4. HBV cccDNA was extracted from PHH using a Hirt DNA extraction method at day 11.

FIG. 3A shows the in vivo liver concentration of SEQ ID NO: 71 and SEQ ID NO: 80 (SEQ ID NO: 71 with 3′ GalNAc) in mice at 24 hours, 72 hours, or 168 hours post administration. FIG. 3B shows the liver Cmax and liver half-life of SEQ ID NO: 71 and SEQ ID NO: 80 in mice. For FIGS. 3A-3B, 10 mg/kg of the indicated oligonucleotide was subcutaneously delivered in C57B1/6 female mice (n=3).

FIG. 4A shows that HBV infected PHH treated with SEQ ID NO: 71 and SEQ ID NO: 72 exhibited an increase in IFN-stimulated gene expression. FIG. 4B shows the level of cytokine induction observed in PBMCs (derived from HBV negative donors) that were contacted with SEQ ID NO: 71, SEQ ID NO: 72, PBS (negative control) and resiquimod (R848) (positive control).

FIGS. 5A-5B show the consensus HBV genome sequence (SEQ ID NO: 100).

### DETAILED DESCRIPTION

Current antiviral therapies inhibit cytoplasmic HBV genomic replication, but are not curative because these therapies do not directly affect nuclear HBV cccDNA, the genomic form that templates viral transcription and sustains viral persistence. Thus, novel approaches that directly target cccDNA regulation are highly desirable. The development of small molecule drugs or therapeutic antibodies that directly target cccDNA is challenging.

The present disclosure provides oligonucleotide compositions that are capable of reducing the expression and/or activity of HBV cccDNA. The oligonucleotides of the present disclosure hybridize to a target sequence at, or in the vicinity of the Enhancer I region of the HBV cccDNA molecule, thereby generating a D-loop at or near the vicinity of the Enhancer I region. While not wishing to be bound by theory, it is believed that generation of the D-loop structure in the cccDNA molecule may act as a cue for the DNA editing and DNA repair machinery in the subject, and may possibly lead to the destruction of the HBV cccDNA (Kasamatsu, H.; Robberson, D. L.; Vinograd, J., Proc Natl Acad Sci. 68 (9): 2252-2257 (1971); Sebesta, M. et al., DNA Repair 12(9): 691-698 (2013)), and may also interfere with the transcription of HBV cccDNA by host RNA polymerase II. In some embodiments, generation of the D-loop structure stimulates innate immunity recognition in a subject infected with HBV cccDNA.

### Definitions

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent, drug, or compound to a subject includes any route of introducing or delivering to a subject a compound to perform its intended function. Administration can be carried out by any suitable route, including orally, intranasally, intrathecally, parenterally (intravenously, intramuscularly, intraperitoneally, or subcutaneously), topically, intrahepatically, transdermally, or any other route described herein. Administration includes self-administration and the administration by another.

As used herein, the terms “amplify” or “amplification” with respect to nucleic acid sequences, refer to methods that increase the representation of a population of nucleic acid sequences in a sample. Nucleic acid amplification methods, such as PCR, isothermal methods, rolling circle methods, etc., are well known to the skilled artisan. See, e.g., Saiki, “Amplification of Genomic DNA” in PCR PROTOCOLS, Innis et al., Eds., Academic Press, San Diego, Calif. 1990, pp 13-20; Wharam et al., Nucleic Acids Res. 2001 Jun. 1; 29(11):E54-E54; Hafner et al., Biotechniques 2001 April; 30(4):852-6, 858, 860 passim. Copies of a particular nucleic acid sequence generated in vitro in an amplification reaction are called “amplicons” or “amplification products”.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-S.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complementary sequence can also be an RNA sequence complementary to the DNA sequence or its complementary sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the “D-loop (displacement loop)” refers to a newly formed triple-stranded region of the Hepatitis B viral genome that is generated by contacting a double-stranded cccDNA molecule of HBV with an oligonucleotide of the present disclosure, where the two strands of the cccDNA molecule are separated for a stretch and held apart by a third strand corresponding to the oligonucleotide of the present disclosure. The third strand has a base sequence which is complementary to one of the strands of the cccDNA and pairs with it, thus displacing the other complementary cccDNA strand in the region. The displaced strand forms the loop of the “D”.

As used herein, the term “effective amount” refers to a quantity sufficient to achieve a desired therapeutic and/or prophylactic effect, e.g., an amount which results in a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

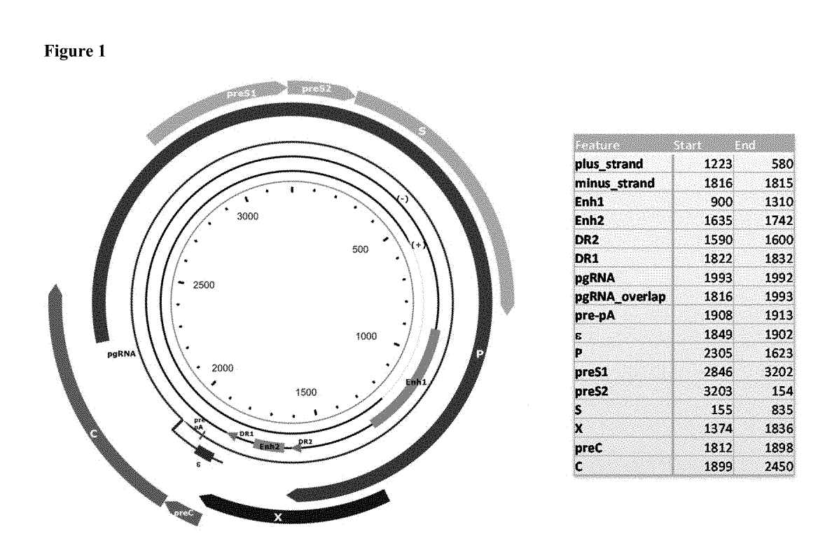

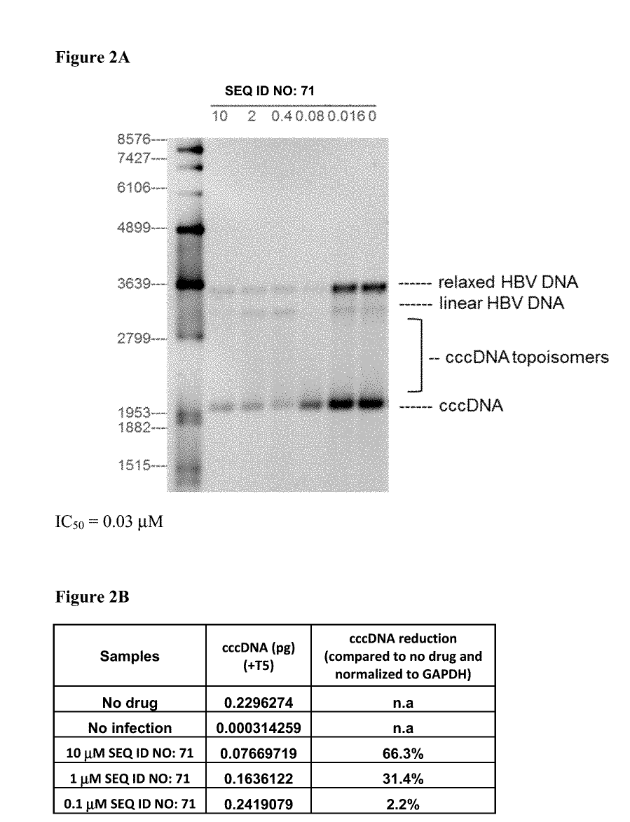

As used herein, “Hepatitis B virus” or “HBV” refers to the well-known non-cytopathic, liver-tropic DNA virus belonging to the Hepadnaviridae family. The HBV genome is partially double-stranded, circular DNA with overlapping reading frames. There are four known genes encoded by the HBV genome, called C, X, P, and S (see FIG. 1). The core protein is encoded by gene C (HBcAg). HBeAg (envelope antigen) is produced by proteolytic processing of the pre-core (pre-C) protein. The HBV DNA polymerase is encoded by gene P. Gene S encodes the surface antigen (HBsAg). The HBsAg gene is one long open reading frame that contains three in frame “start” (ATG) codons that divide the gene into three regions, pre-S1, pre-S2, and S. Because of the multiple start codons, polypeptides of three different sizes called large (L), middle (M), and small (S) (pre-S1+pre-S2+S, pre-S2+S, or S respectively) are produced. Gene X encodes a decoy protein that permits HBsAg in the blood to sequester anti-HBsAg antibodies and allow infectious viral particles to escape immune detection. Eight genotypes of HBV, designated A to H, have been identified, each having a distinct geographical distribution. The term “HBV” includes any of the eight genotypes of HBV (A to H). The term “HBV,” as used herein, also refers to naturally occurring DNA sequence variations of the HBV genome.

As used herein, the terms “Hepatitis B virus-associated disease” or “HBV-associated disease,” refer to a disease or disorder that is caused by, or associated with HBV infection and/or replication, including acute hepatitis B, acute fulminant hepatitis B, chronic hepatitis B, liver failure, end-stage liver disease, cirrhosis, and hepatocellular carcinoma.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” can be an individual organism, a vertebrate, a mammal, or a human. In a preferred embodiment, the individual, patient or subject is a human.

The term “modification” in the context of an oligonucleotide includes but is not limited to (a) end modifications, e.g., 5′ end modifications or 3′ end modifications, (b) nucleobase (or “base”) modifications, including replacement or removal of bases, (c) sugar modifications, including modifications at the 2′, 3′, and/or 4′ positions, and (d) backbone modifications, including modification or replacement of the phosphodiester linkages.

The term “modified nucleotide” generally refers to a nucleotide having a modification to the chemical structure of one or more of the base, the sugar, and the phosphodiester linkage or backbone portions, including nucleotide phosphates.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides of the method which function as primers or probes are generally at least about 10-15 nucleotides long and more preferably at least about 14 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

“Probe” as used herein refers to a nucleic acid that interacts with a target nucleic acid via hybridization. A probe may be fully complementary to a target nucleic acid sequence or partially complementary. The level of complementarity will depend on many factors based, in general, on the function of the probe. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. Probes are typically at least about 10, 15, 20, 25, 30, 35, 40, 50, 60, 75, 100 nucleotides or more in length.

As used herein, the term “sample” refers to clinical samples obtained from a patient. In preferred embodiments, a sample is obtained from a biological source (i.e., a “biological sample”), such as tissue, or bodily fluid collected from a subject. Sample sources include, but are not limited to, stool, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material).

The term “sense strand” as used herein means the strand of double-stranded DNA (dsDNA) that includes at least a portion of a coding sequence of a functional protein. “Anti-sense strand” means the strand of dsDNA that is the reverse complement of the sense strand.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5× Denhart's solution at 42° C. overnight; washing with 2× SSC, 0.1% SDS at 45° C.; and washing with 0.2× SSC, 0.1% SDS at 45° C. In another example, stringent hybridization conditions should not allow for hybridization of two nucleic acids which differ over a stretch of 20 contiguous nucleotides by more than two bases.

The term “substantially complementary” as used herein means that two sequences hybridize under stringent hybridization conditions. The skilled artisan will understand that substantially complementary sequences need not hybridize along their entire length. In particular, substantially complementary sequences may comprise a contiguous sequence of bases that do not hybridize to a target sequence, positioned 3′ or 5′ to a contiguous sequence of bases that hybridize under stringent hybridization conditions to a target sequence.

As used herein, the term “target sequence” refers to a nucleic acid sequence of interest that is present in a sample and is capable of hybridizing to an oligonucleotide of the present disclosure. In some embodiments, the target sequence is at, or in the vicinity of the Enhancer I region of an HBV cccDNA molecule. In a further embodiment, hybridization of the target sequence to an oligonucleotide of the present disclosure results in the destruction of the HBV cccDNA molecule by the host DNA repair mechanism.

“Treating” or “treatment” as used herein covers the treatment of a disease or condition (e.g., HBV infection and/or an HBV-associated disorder) in a subject, such as a human, and includes: (i) reducing the occurrence or inhibiting a disease or condition, i.e., arresting its development; (ii) relieving a disease or condition, i.e., causing regression of the disease or condition; (iii) slowing progression of the disease or condition; and/or (iv) inhibiting, relieving, delaying the onset, or slowing progression of one or more symptoms of the disease or condition. In some embodiments, treatment results in the complete cure of HBV infection and/or an HBV-associated disorder.

The HBV infection or the HBV-associated disorder may be caused by one or more HBV genotypes such as HBV genotype A, HBV genotype B, HBV genotype C, HBV genotype D, HBV genotype E, HBV genotype F, HBV genotype G, or HBV genotype H. In some embodiments, the HBV-associated disorder is chronic hepatitis B, liver failure, cirrhosis, or hepatocellular carcinoma. In certain embodiments, the subject is human.

In some embodiments of the method, the subject displays elevated levels of HBV cccDNA compared to a normal control subject. In certain embodiments, treatment with the oligonucleotide reduces levels of HBV cccDNA in the subject. Additionally or alternatively, in some embodiments of the method, the subject displays elevated liver levels of HBV cccDNA compared to a normal control subject. In certain embodiments, treatment with the oligonucleotide reduces liver levels of HBV cccDNA in the subject. In any of the above embodiments, the HBV cccDNA levels are reduced for about 1 hour to about 80 hours following administration of the oligonucleotide.

Symptoms associated with HBV infection and/or an HBV-associated disorder include, but are not limited to the presence of liver HBV cccDNA, the presence of serum and/or liver HBV antigen (e.g., HBsAg and/or HBeAg), elevated ALT, elevated AST, the absence or low level of anti-HBV antibodies, liver injury, cirrhosis, delta hepatitis, acute hepatitis B, acute fulminant hepatitis B, chronic hepatitis B, liver fibrosis, end-stage liver disease, hepatocellular carcinoma, serum sickness-like syndrome, anorexia, nausea, vomiting, low-grade fever, myalgia, fatigability, disordered gustatory acuity and smell sensations (aversion to food and cigarettes), right upper quadrant and epigastric pain (intermittent, mild to moderate), hepatic encephalopathy, somnolence, disturbances in sleep pattern, mental confusion, coma, ascites, gastrointestinal bleeding, coagulopathy, jaundice, hepatomegaly (mildly enlarged, soft liver), splenomegaly, palmar erythema, spider nevi, muscle wasting, spider angiomas, vasculitis, variceal bleeding, peripheral edema, gynecomastia, testicular atrophy, abdominal collateral veins (caput medusa), high levels of alanine aminotransferase (ALT) and aspartate aminotransferase (AST) (within a range of 1000-2000 IU/mL), ALT levels higher than AST levels, elevated gamma-glutamyl transpeptidase (GGT) and/or alkaline phosphatase (ALP) levels, decreased albumin levels, elevated serum iron levels, leukopenia (i.e granulocytopenia), lymphocytosis, increased erythrocyte sedimentation rate (ESR), shortened red blood cell survival, hemolysis, thrombocytopenia, a prolongation of the international normalized ratio (INR), the presence of serum HBV DNA, elevation of the aminotransferases (<5 times the ULN), increased bilirubin levels, prolonged prothrombin time (PT), hyperglobulinemia, the presence of tissue-nonspecific antibodies, such as anti-smooth muscle antibodies (ASMAs) or antinuclear antibodies (ANAs), the presence of tissue-specific antibodies, such as antibodies against the thyroid gland, elevated levels of rheumatoid factor (RF), hyperbilirubinemia, low platelet and white blood cell counts, AST levels higher than ALT levels, lobular inflammation accompanied by degenerative and regenerative hepatocellular changes, and predominantly centrilobular necrosis. “Treatment” can also mean prolonging survival as compared to expected survival in the absence of treatment.

It is also to be appreciated that the various modes of treatment of the diseases or conditions described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Oligonucleotide Compositions of the Present Disclosure

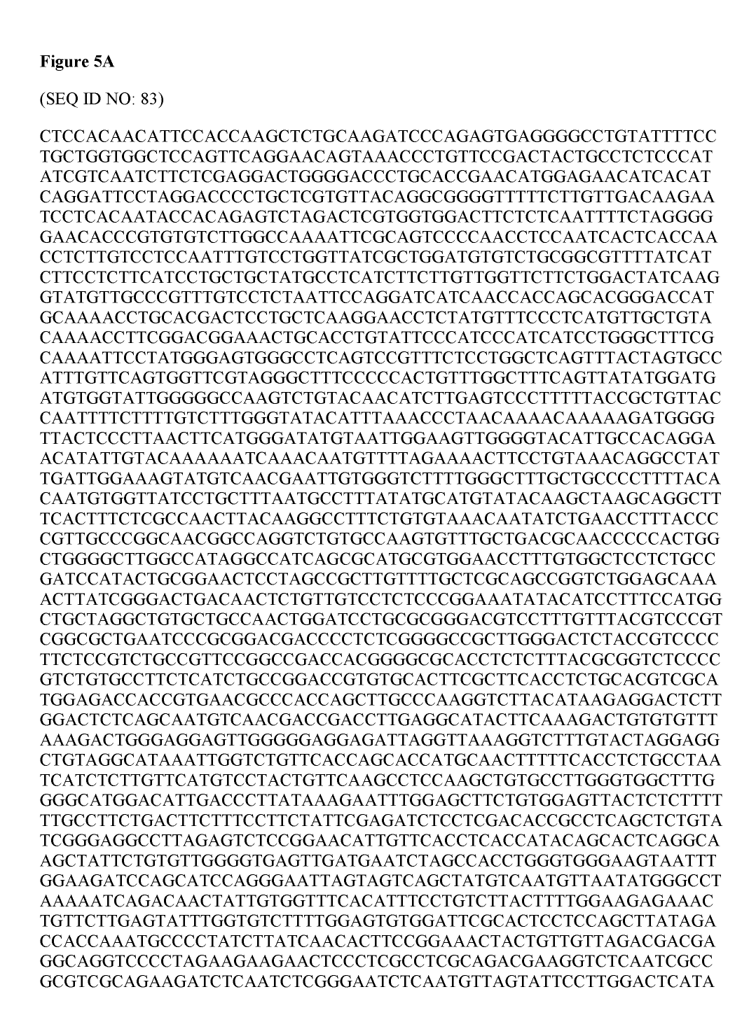

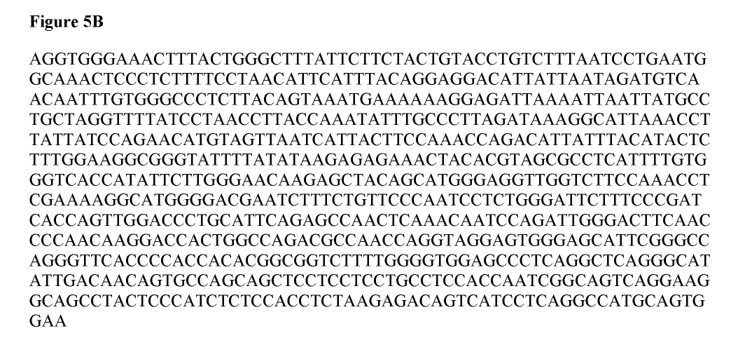

The present disclosure provides oligonucleotides and oligonucleotide compositions that are capable of reducing the expression and/or activity of HBV cccDNA. The oligonucleotides of the present disclosure hybridize to a target sequence at, or in the vicinity of the Enhancer I region of the HBV cccDNA molecule, thereby generating a D-loop at or near the vicinity of the Enhancer I region. FIG. 1 shows that the nucleotide sequence of the Enhancer I region starts at nucleotide position 900 and ends at nucleotide position 1310, and FIGS. 5A-5B is a consensus HBV genome sequence.

The oligonucleotides of the present disclosure target a region of an HBV cccDNA genome consisting of nucleotide position 900-1310 (Enhancer I region) with an oligonucleotide that is at least 90% complementary to the target region of the HBV cccDNA.

In some embodiments, the oligonucleotides of the present disclosure target an HBV DNA sequence that is within the Enhancer I region (i.e., a target nucleotide sequence located between and including nucleotide positions 900 and 1310 of the HBV genome).

In some embodiments, the oligonucleotides of the present disclosure target an HBV DNA sequence that is no more than 50 base pairs, no more than 45 base pairs, no more than 40 base pairs, no more than 35 base pairs, no more than 30 base pairs, no more than 25 base pairs, no more than 20 base pairs, no more than 15 base pairs, no more than 10 base pairs, or no more than 5 base pairs upstream of the Enhancer I region. In certain embodiments, the oligonucleotides of the present disclosure target an HBV DNA sequence that is no more than 50 base pairs, no more than 45 base pairs, no more than 40 base pairs, no more than 35 base pairs, no more than 30 base pairs, no more than 25 base pairs, no more than 20 base pairs, no more than 15 base pairs, no more than 10 base pairs, or no more than 5 base pairs downstream of the Enhancer I region.

In some embodiments, the oligonucleotides of the present disclosure target an HBV DNA sequence that is located anywhere between position 969 and position 987 of the HBV genome. In certain embodiments, the oligonucleotides of the present disclosure target an HBV DNA sequence that is located anywhere between position 1094 and position 1116 of the HBV genome. In some embodiments, the oligonucleotides of the present disclosure target an HBV DNA sequence that is located anywhere between position 1136 and position 1155 of the HBV genome. In some embodiments, the oligonucleotides of the present disclosure target an HBV DNA sequence that is located anywhere between position 1174 and position 1194 of the HBV genome. In other embodiments, the oligonucleotides of the present disclosure target an HBV DNA sequence that is located anywhere between position 1194 and position 1216 of the HBV genome. In some embodiments, the oligonucleotides of the present disclosure target an HBV DNA sequence that is located anywhere between position 1297 and position 1315 of the HBV genome.

Examples of oligonucleotides of the present disclosure are presented in Table 1.

TABLE 1

| SEQ ID | |
|---|---|
| NO: | Nucleobase Sequence (5′-3′) |
| 1 | AAGCCCCAGCCAGUGGGGGUU |
| 2 | CCAAGCCCCAGCCAGUGGG |
| 3 | CUUGUAAGUUGGCGAGAAAG |
| 4 | UACUUUCCAAUCAAUAGGC |
| 5 | CCUAUUGAUUGGAAAGU |
| 6 | GCCUAUUGAUUGGAAAGUA |
| 7 | UGAACCUUUACCCCGUUGCC |
| 8 | UGCGUCAGCAAACACUUGGC |
| 9 | GCGUCAGCAAACACUUGGCA |
| 10 | UCUCGCCAACUUACAAGGCC |
| 11 | AUUGAUUGGAAAGU |
| 12 | GCCAAGUGUUUGCUGACGC |
| 13 | GCUCGCAGCCGGUCUGGAG |
| 14 | ACUUUCCAAUCAAU |
| 15 | GGCAACGGGGUAAAGGUUCA |
| 16 | GCCGGGCAACGGGGUAAAGG |
| 17 | GCGUCAGCAAACACUUGGC |
| 18 | CCACGCAUGCGCUGAUGGCC |
| 19 | CCAGCCAGUGGGGGUUGCGUC |
| 20 | GCCCCCAGCCAGUGGGGGUU |
| 21 | AAGCCCCAGCCAGUGGGGG |
| 22 | AGCCAGUGGGGGUUGCGUC |
| 23 | UUCCACGCAUGCGCUGAUGG |
| 24 | AAAGGUUCCACGCAUGCGCA |
| 25 | UUCCGCAGUAUGGAUCGGC |
| 26 | CGCAGUAUGGAUCGGCAGAGG |
| 27 | AGGAGUUCCGCAGUAUGGAUC |
| 28 | GGCUGCGAGCAAAACAAGC |
| 29 | CCGGCUGCGAGCAAAACAAGC |
| 30 | CCGGCUGCGAGCAAAACAA |
| 31 | CCAGACCGGCUGCGAGCAAAA |
| 32 | CUCCAGACCGGCUGCGAGC |
| 33 | GCUCCAGACCGGCUGCGAGC |
| 34 | CCGGCUGCGAGCAAAACAAG |
| 35 | UUUGCUCCAGACCGGCUGCG |
| 36 | UGCUCCAGACCGGCUGCGAG |
| 37 | ACCGGCUGCGAGCAAAACAA |
| 38 | GUUGCCGGGCAACGGGGUAA |
| 39 | UUGCCGGGCAACGGGGUAAA |
| 40 | GCCGGGCAACGGGGUAAAGG |
| 41 | CCGGGCAACGGGGUAAAGGU |
| 42 | CGGGCAACGGGGUAAAGGUU |
| 43 | CAGUGGGGGUUGCGUCAGCA |
| 44 | AGUGGGGGUUGCGUCAGCAA |
| 45 | GUGGGGGUUGCGUCAGCAAA |
| 46 | UGGGGGUUGCGUCAGCAAAC |
| 47 | GGGGGUUGCGUCAGCAAACA |
| 48 | GGGGUUGCGUCAGCAAACAC |
| 49 | GGUUGCGUCAGCAAACACUU |
| 50 | UCGCCAACUUACAAGGCCUU |
| 51 | CUCGCCAACUUACAAGGCCU |
| 52 | UUCUCGCCAACUUACAAGGC |
| 53 | UUUCUCGCCAACUUACAAGG |
| 54 | CUUUCUCGCCAACUUACAAG |
| 55 | ACUUUCUCGCCAACUUACAA |
| 56 | UGAACCUUUACCCCGUUGC |
| 57 | CCUUUACCCCGUUGCCCGGC |
| 58 | GAUCCAUACUGCGGAACUCCU |
| 59 | GCUUGUUUUGCUCGCAGCC |
| 60 | GCUUGUUUUGCUCGCAGCCGG |
| 61 | UUGUUUUGCUCGCAGCCGG |
| 62 | UUUUGCUCGCAGCCGGUCU |
| 63 | UUUUGCUCGCAGCCGGUCUGG |
| 64 | GCUCGCAGCCGGUCUGGAGC |
| 65 | CUUGUUUUGCUCGCAGCCGG |

In certain embodiments, SEQ ID NOs: 1-65 are modified, for example, as described in this disclosure.

In some embodiments of the oligonucleotides of the present disclosure, one or more nucleobases of any of SEQ ID NOs: 1-65 or complements thereof, may be substituted with a modified nucleobase selected from among adenine (A), guanine (G), thymine (T), cytosine (C), uracil (U), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 3′-amino-2′-deoxy-2,6-Diaminopurine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine, 7-deazaguanosine, 2-aminopyridine and 2-pyridone.

In some embodiments of the oligonucleotides of the present disclosure, one or more nucleobases of any of SEQ ID NOs: 1-65 or complements thereof, may be substituted with a modified nucleobase selected from among tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-am-oe1hoxy)-H-pyrimido[5,4-b][1,4]benzoxazin-2(3H)-one), carbazole cytidine (2H-pyrimido[4,5-b]indol-2-one), pyridoindole cytidine (H-pyrido[3,2 ,5]pyrrolo[2,3-d]pyrimidin-2-one).

Additionally or alternatively, in some embodiments, the sugars of one or more nucleobases of any of SEQ ID NOs: 1-65 or complements thereof, may be substituted with modified sugars selected from among 2′-OH (ribose) nucleosides, 2′-O-Methylated (2′-O-Me) nucleosides, 2′-O-methoxyethyl (2′-MOE) nucleosides, 2′-ribo-F nucleosides, 2′-arabino-F nucleosides, 2′-Me nucleosides, and 2′-Me-2′-F nucleosides. In some embodiments, the sugars of one or more nucleobases of any of SEQ ID NOs: 1-65 or complements thereof, may be substituted with modified sugars selected from among 2′-F and 2′-O-alkyl, wherein said O-alkyl is optionally substituted with alkoxy.

Additionally or alternatively, in some embodiments, the original backbone linkage of one or more nucleobases of any of SEQ ID NOs: 1-65 or complements thereof, may be replaced with an alternate intersubunit linkage selected from among phosphodiester intersubunit linkages, thiophosphate intersubunit linkages, phosphoramidate intersubunit linkages, and thiophosphoramidate intersubunit linkages.

In some embodiments, one or more of the nucleotides includes modification of the 2′ position of the sugar ring or modification of the internucleotide subunit linkage. For example, some embodiments include one or more 2′-F or 2′-O-alkyl, wherein said O-alkyl is optionally substituted with alkoxy, e.g., some embodiments include a 2′-OMe and/or 2′-F modification. In some embodiments, one or more of the internucleotide subunit linkages is a thiophosphate linkage. In some embodiments, one or more of the internucleotide subunit linkages is a phosphoramidate linkage. In some embodiments, one or more of the internucleotide subunit linkages is a thiophosphoramidate linkage.

In some embodiments, the oligonucleotides of the present disclosure include modified nucleotides. For example, compounds of the present disclosure may include nucleotides of Formula (I):

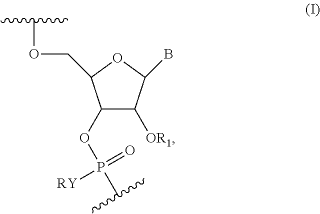

wherein R is H or a positively charged counter ion, B is independently in each instance a natural or an unmodified nucleobase or a modified nucleobase, Y is O or S, R₁ is —(CR′₂)₂OCR′₃, and R′ is independently in each instance H or F.

In nucleotides of Formula (I), R₁ is —(CR′₂)₂OCR′₃. In some embodiments, R′ is H in each instance. In other embodiments, at least one R′ is F, for example, 1, 2, 3, 4, 5, 6, or 7 R's are F. In some embodiments, CR′₃ contains 1, 2 or 3 F moieties. For example, in embodiments, R₁ is selected from the group consisting of —CH₂CH₂OCH₃ (or MOE), —CF₂CH₂OCH₃, —CH₂CF₂OCH₃, —CH₂CH₂OCF₃, —CF₂CF₂OCH₃, —CH₂CF₂OCF₃, —CF₂CH₂OCF₃, —CF₂CF₂OCF₃, —CHFCH₂OCH₃, —CHFCHFOCH₃, —CHFCH₂OCFH₂, —CHFCH₂OCHF₂ and —CH₂CHFOCH₃.

In embodiments, the nucleotide of Formula I is

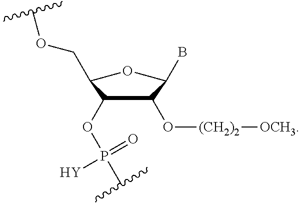

In embodiments, compounds of the present disclosure include at least one nucleotide of Formula (II):

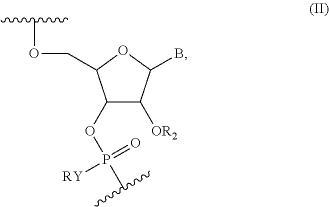

wherein Y is S or O, R is H or a positively charged counter ion, B is a nucleobase, R₂ is —CR′₃, —CR′₂OCR′₃, —(CR′₂)₃OCR′₃ or —(CR′₂)₁₋₂CR′₃, or R₂ is —(CR′₂)₂OCR′₃ and Y is O and R′ is independently in each instance H or F.

In the nucleotide of Formula (II), R₂ is —CR′₃, —(CR′₂)₁₋₃OCR′₃, or —(CR′₂)₁₋₂CR′₃. In some embodiments, R₂ is —CR′₃ or —CR′₂CR′₃. In some embodiments, R′ is H in each instance. In other embodiments, at least one R′ is F, for example, 1, 2, 3, 4, or 5 R's are F. In some embodiments, CR′₃ contains 1, 2 or 3 F moieties. For example, in embodiments, R₁ is selected from the group consisting of —CH₃ (or Me), —CFH₂, —CHF₂, CF₃, —CH₂OCH₃, —CFH₂OCH₃, —CHF₂OCH₃, —CF₃OCH₃, —CH₂OCFH₂, —CH₂OCHF₂, —CH₂OCF₃, —CFH₂OCH₃, —CFH₂OCFH₂, —CFH₂OCHF₂, —CFH₂OCF₃, —CHF₂OCH₃, —CHF₂OCFH₂, —CHF₂OCHF₂, —CHF₂OCF₃, —(CR′₂)₃OCR′₃, —CH₂CH₃ (or Et), —CFH₂CH₃, —CHF₂CH₃, —CF₃CH₃, —CH₂CFH₂, —CH₂CHF₂, —CH₂CF₃, —CFH₂CH₃, —CFH₂CFH₂, —CFH₂CHF₂, —CFH₂CF₃, —CHF₂CH₃, —CHF₂CFH₂, —CHF₂CHF₂, —CHF₂CF₃, —CH₂CH₂CH₃, CF₂CH₂CH₃, CH₂CF₂CH₃, CH₂CH₂CF₃, CF₂CF₂CH₃, CH₂CF₂CF₃, CF₂CH₂CF₃, CF₂CF₂CF₃, CHFCH₂CH₃, CHFCHFOCH₃, CHFCH₂CFH₂, CHFCH₂CHF₂ and CH₂CHFCH₃. In embodiments, R₁ is —CH₃ (or Me) or —CH₂CH₃ (or Et). In embodiments, the nucleotides of Formula II are selected from the group consisting of

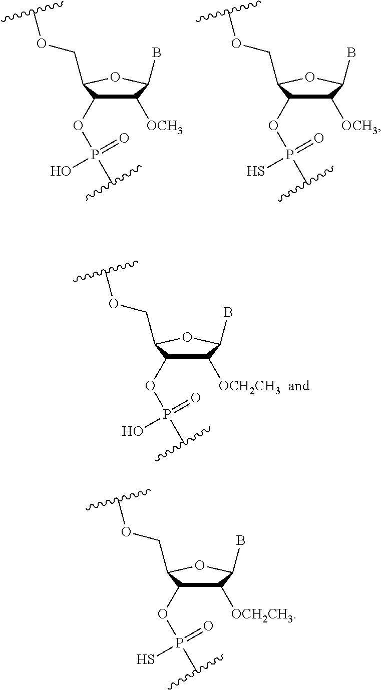

In compounds of Formulae (I) or (II), Y may be O or S. In some embodiments, Y is S in at least one instance (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.). In other embodiments, Y is S in at least one instance and O in at least another instance. In other embodiments, Y is S in each instance. In some embodiments, Y is O in at least one instance (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.).

The disclosed oligonucleotides comprise at least one nucleotide of Formula (I). In embodiments, the disclosed oligonucleotides comprise 1, 2, 3, 4, 5, 6, 7, 8, 9, 10, 11, 12, 13, 14, 15, 16, 17, 18, 19, 20, 21, 22, 23, 24 nucleotides of Formula (I). In embodiments, the disclosed oligonucleotides comprise 1, 2, 3, 4, 5, 6, 7, 8, 9, 10, 11, 12, 13, 14, 15, 16, 17, 18, 19, 20, 21, 22, 23, 24 nucleotides of Formula (II). In some embodiments, the oligonucleotide comprises from 2 to 40 nucleotides, for example, 8 to 26 nucleotides or integers there between.

In embodiments where more than one nucleotide of Formula (I) are included, the nucleotide may be the same or different. In some embodiments one or more nucleotides of Formula (II) are included, and may be the same or different. For example, in some embodiments, the oligonucleotide comprises at least one nucleotide of Formula (I) and at least one nucleotide of Formula (II). In some embodiments, the oligonucleotide comprises at least one nucleotide of Formula (I), wherein at least one R₁ is MOE and at least one nucleotide of Formula (II), wherein R₂ is Me or Et. In some embodiments, the oligonucleotide comprises at least 2 alternating nucleotides of Formula (I) and Formula (II). For example, 2, 3, 4, 5, 6, 7, 8, 9, 10, 11, 12, 13, 14, 15, 16, 17, 18, 19, 20, 21, 22, 23, 24 nucleotides with alternating 2′ modification (e.g., Me-MOE-Me-MOE . . . or Et-MOE-Et-MOE-Et-MOE . . . ).

In some embodiments, the oligonucleotide comprises a 2′-fluoronucleotide of the Formula (IIIa) and/or (IIIb):

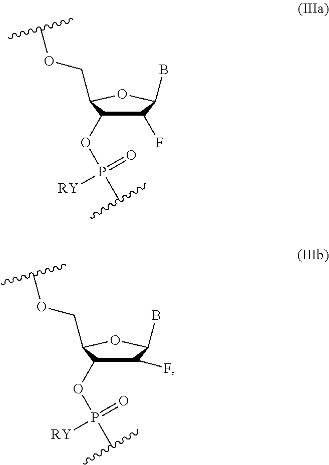

where Y is S or O, R is H or a positively charged counter ion, and B is a nucleobase.

In some embodiments, the oligonucleotide comprises at least 4 alternating nucleotides of Formulae (I) or (II) and (IIIa). For example, the oligonucleotide comprises 4, 5, 6, 7, 8, 9, 10, 11, 12, 13, 14, 15, 16, 17, 18, 19, 20, 21, 22, 23, 24 alternating nucleotides.

The nucleobases, B, of the nucleotides of Formulae (I), (II), (IIIa), and (IIIb) may each independently be a natural or an unmodified nucleobase or a modified nucleobase. In some embodiments, the modified nucleotides include 2,6-diaminopurine nucleobases, but optionally not adenine. In some embodiments, the modified nucleotides include 5-methyluracil nucleobases, but optionally not uracil. In some embodiments, the modified nucleotides include 2,6-diaminopurine nucleobases, but not adenine and 5-methyluracil nucleobases, but optionally not uracil.

Y in each nucleotide of Formulae (I), (II), (IIIa), and (IIIb) may be independently O or S. In some embodiments, Y is S in at least one instance (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.). In other embodiments, Y is S in at least one instance and O in at least another instance. In other embodiments, Y is S in each instance. In some embodiments, Y is O in at least one instance (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.).

In embodiments where more than one nucleotide of each of Formulae (I), (II), (IIIa), and (IIIb) are included, the more than one nucleotides such Formulae may be the same or different. For example, in some embodiments, the nucleotide comprises at least one nucleotide of Formulae (I) (II), (IIIa), and (IIIb) in addition to at least one nucleotide of Formula (I). In some embodiments, the nucleotide comprises at least 2 alternating nucleotides of Formula (I) and/or Formula (II) and/or (III). For example, disclosed oligonucleotides may include 2, 3, 4, 5, 6, 7, 8, 9, 10, 11, 12, 13, 14, 15, 16, 17, 18, 19, 20, 21, 22, 23, 24 nucleotides with alternating 2′ modifications.

In some embodiments, the oligonucleotides of the present disclosure contain one or more of the above modifications, and have a nucleobase sequence having an affinity for nuclear HBV cccDNA or hybridize to a target sequence at, or in the vicinity of the Enhancer I region of the HBV cccDNA molecule, thereby generating/maintaining a D-loop at or near the vicinity of the Enhancer I region. For example, in some embodiments, the oligonucleotides of the present disclosure contain one or more of the above modifications and a nucleobase sequence according to one of the sequences listed herein.

In some embodiments, the oligonucleotides of the present technology include modified nucleotides. For example, compounds of the present disclosure include nucleotides of Formula (I):

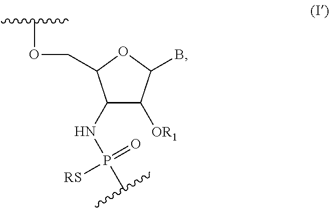

wherein R is H or a positively charged counter ion, B is independently in each instance a natural or an unmodified nucleobase or a modified nucleobase, R₁ is —(CR′₂)₂OCR′₃, and R′ is independently in each instance H or F

In nucleotides of Formula (I′), R₁ is —(CR′₂)₂OCR′₃. In some embodiments, R′ is H in each instance. In other embodiments, at least one R′ is F, for example, 1, 2, 3, 4, 5, 6, or 7 R's are F. In some embodiments, CR′₃ contains 1, 2 or 3 F moieties. For example, in embodiments, R₁ is selected from the group consisting of —CH₂CH₂OCH₃ (or MOE), —CF₂CH₂OCH₃, —CH₂CF₂OCH₃, —CH₂CH₂OCF₃, —CF₂CF₂OCH₃, —CH₂CF₂OCF₃, —CF₂CH₂OCF₃, —CF₂CF₂OCF₃, —CHFCH₂OCH₃, —CHFCHFOCH₃, —CHFCH₂OCFH₂, —CHFCH₂OCHF₂ and —CH₂CHFOCH₃. In embodiments, the nucleotide of Formula I is

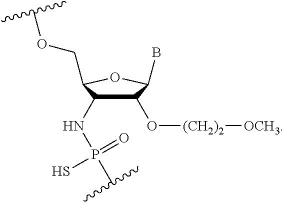

In embodiments, compounds of the present disclosure include at least one nucleotide of Formula (I′) and/or at least one nucleotide of Formula (II′):

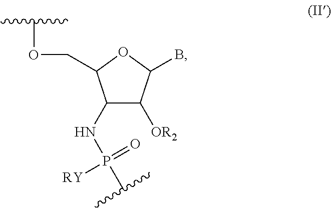

wherein Y is S or O, R is H or a positively charged counter ion, B is a nucleobase, R₂ is —CR′₃, —CR′₂OCR′₃, —(CR′₂)₃OCR′₃ or —(CR′₂)₁₋₂CR′₃, or R₂ is —(CR′₂)₂OCR′₃ and Y is O and R′ is independently in each instance H or F.

In the nucleotide of Formula (II′), R₂ is —CR′₃, —(CR′₂)₁₋₃OCR′₃, or —(CR′₂)₁₋₂CR′₃. In some embodiments, R₂ is —CR′₃ or —CR′₂CR′₃. In some embodiments, R′ is H in each instance. In other embodiments, at least one R′ is F, for example, 1, 2, 3, 4, or 5 R's are F. In some embodiments, CR′₃ contains 1, 2 or 3 F moieties. For example, in embodiments, R₁ is selected from the group consisting of —CH₃ (or Me), —CFH₂, —CHF₂, CF₃, —CH₂OCH₃, —CFH₂OCH₃, —CHF₂OCH₃, —CF₃OCH₃, —CH₂OCFH₂, —CH₂OCHF₂, —CH₂OCF₃, —CFH₂OCH₃, —CFH₂OCFH₂, —CFH₂OCHF₂, —CFH₂OCF₃, —CHF₂OCH₃, —CHF₂OCFH₂, —CHF₂OCHF₂, —CHF₂OCF₃, —(CR′₂)₃OCR′₃, —CH₂CH₃ (or Et), —CFH₂CH₃, —CHF₂CH₃, —CF₃CH₃, —CH₂CFH₂, —CH₂CHF₂, —CH₂CF₃, —CFH₂CH₃, —CFH₂CFH₂, —CFH₂CHF₂, —CFH₂CF₃, —CHF₂CH₃, —CHF₂CFH₂, —CHF₂CHF₂, —CHF₂CF₃, —CH₂CH₂CH₃, CF₂CH₂CH₃, CH₂CF₂CH₃, CH₂CH₂CF₃, CF₂CF₂CH₃, CH₂CF₂CF₃, CF₂CH₂CF₃, CF₂CF₂CF₃, CHFCH₂CH₃, CHFCHFOCH₃, CHFCH₂CFH₂, CHFCH₂CHF₂ and CH₂CHFCH₃. In embodiments, R₁ is —CH₃ (or Me) or —CH₂CH₃(or Et). In embodiments, the nucleotides of Formula II are selected from the group consisting of

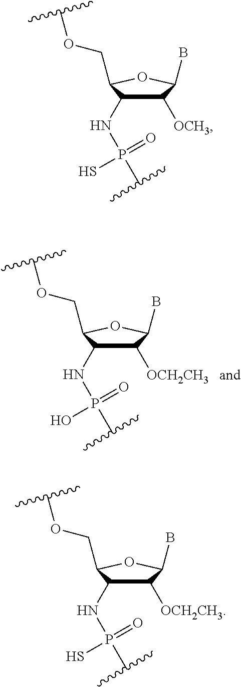

In compounds of Formulae (I′) or (II′), Y may be O or S. In some embodiments, Y is S in at least one instance (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.). In other embodiments, Y is S in at least one instance and O in at least another instance. In other embodiments, Y is S in each instance. In some embodiments, Y is O in at least one instance (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.).

The disclosed oligonucleotides comprise at least one nucleotide of Formula (I′). In embodiments, the disclosed oligonucleotides comprise 1, 2, 3, 4, 5, 6, 7, 8, 9, 10, 11, 12, 13, 14, 15, 16, 17, 18, 19, 20, 21, 22, 23, 24 nucleotides of Formula (I′). In embodiments, the disclosed oligonucleotides comprise 1, 2, 3, 4, 5, 6, 7, 8, 9, 10, 11, 12, 13, 14, 15, 16, 17, 18, 19, 20, 21, 22, 23, 24 nucleotides of Formula (II′). In some embodiments, the oligonucleotide comprises from 2 to 40 nucleotides, for example, 8 to 26 nucleotides or integers there between.

In embodiments where more than one nucleotide of Formula (I′) are included, the nucleotide may be the same or different. In some embodiments one or more nucleotides of Formula (II′) are included, and may be the same or different. For example, in some embodiments, the oligonucleotide comprises at least one nucleotide of Formula (I′) and at least one nucleotide of Formula (II′). In some embodiments, the oligonucleotide comprises at least one nucleotide of Formula (I′), wherein at least one R₁ is MOE and at least one nucleotide of Formula (II′), wherein R₂ is Me or Et. In some embodiments, the oligonucleotide comprises at least 2 alternating nucleotides of Formula (I′) and Formula (II). For example, 2, 3, 4, 5, 6, 7, 8, 9, 10, 11, 12, 13, 14, 15, 16, 17, 18, 19, 20, 21, 22, 23, 24 nucleotides with alternating 2′ modification (e.g., Me-MOE-Me-MOE . . . or Et-MOE-Et-MOE-Et-MOE . . . ).

In some embodiments, the nucleotide of Formula (I′) and/or Formula (II′) may be included, and is represented by the following:

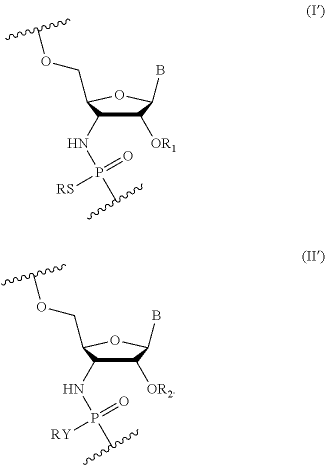

In some embodiments, the oligonucleotide comprising the nucleotide of Formula (I) and/or comprises a 2′-fluoronucleotide of the Formula (IIIa′) and/or (IIIb′):

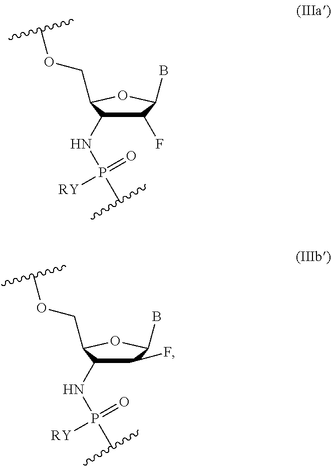

where Y is S or O, R is H or a positively charged counter ion, and B is a nucleobase.

In some embodiments, the oligonucleotide comprises at least 4 alternating nucleotides of Formulae (I′) and (IIIa′). For example, the oligonucleotide comprises 4, 5, 6, 7, 8, 9, 10, 11, 12, 13, 14, 15, 16, 17, 18, 19, 20, 21, 22, 23, 24 alternating nucleotides.

Certain embodiments include an oligonucleotide comprising 4-40 nucleotides, and comprising Formula (IV′):

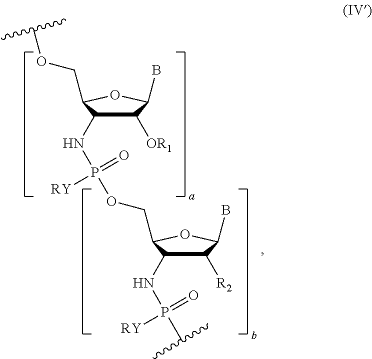

wherein Y is S or O, R is H or a positively charged counter ion, B is a nucleobase, R₁ is —(CR′₂)₂OCR′₃, R₂ is selected from —OCR′₃, —OCR′₂OCR′₃, —O(CR′₂)₃OCR′₃ or —O(CR′₂)₁₋₂CR′₃ and F, R′ is independently in each instance H or F, and a is an integer of 1-10 and b is an integer from 1-10, where the to 20, e.g., 1, 2, 3, 4, 5, 6, 7, 8, 9, 10, 11, 12, 13, 14, 15, 16, 17, 18, 19 and 20.

Compounds of the present disclosure include compounds comprising the following Formula (III′):

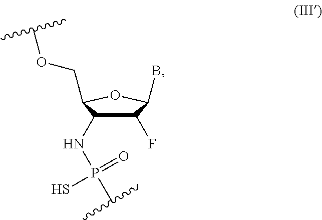

wherein Y is S or O, R is H or a positively charged counter ion, and B is independently in each instance a natural or an unmodified nucleobase or a modified nucleobase; and optionally comprising one or more of formula (I′), (II′) and/or (IV′).

The nucleobases, B, of the nucleotides of Formulae (I′), (II′), (IIIa′), (IIIb′), (IV′) and (V′) may each independently be a natural or an unmodified nucleobase or a modified nucleobase. In some embodiments, the modified nucleotides include 2,6-diaminopurine nucleobases, but optionally not adenine. In some embodiments, the modified nucleotides include 5-methyluracil nucleobases, but optionally not uracil. In some embodiments, the modified nucleotides include 2,6-diaminopurine nucleobases, but not adenine and 5-methyluracil nucleobases, but optionally not uracil.

Y in each nucleotide of Formulae (II′), (IIIa′), (IIIb′), (IV′) and (V′) may be independently O or S. In some embodiments, Y is S in at least one instance (e.g., 1, 2, 3, 4, 5, 6,7, 8, 9, 10, 11, 12, 13, 14, 15, 16, 17, 18, 19, 20, 21, 22, 23, 24, 25, 26, 27, 28, 29, 30 etc.). In other embodiments, Y is S in at least one instance and O in at least another instance. In other embodiments, Y is S in each instance. In some embodiments, Y is O in at least one instance (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.).

In embodiments where more than one nucleotide of each of Formulae (I′), (II′), (IIIa′), (IIIb′), (IV′) and (V′) are included, the more than one nucleotides such Formulae may be the same or different. For example, in some embodiments, the nucleotide comprises at least one nucleotide of Formula (II′), (III′), (IV′) , (V′) and/or (V′) in addition to at least one nucleotide of Formula (I). In some embodiments, the nucleotide comprises at least 2 alternating nucleotides of Formula (I′) and/or Formula (II′) and/or (III′) and/or (IV′) , (V′) and/or (V′). For example, disclosed oligonucleotides may include 2, 3, 4, 5, 6, 7, 8, 9, 10, 11, 12, 13, 14, 15, 16, 17, 18, 19, 20, 21, 22, 23, 24 nucleotides with alternating 2′ modifications.

In embodiments, the nucleotides of the oligonucleotide are selected from the group consisting of:

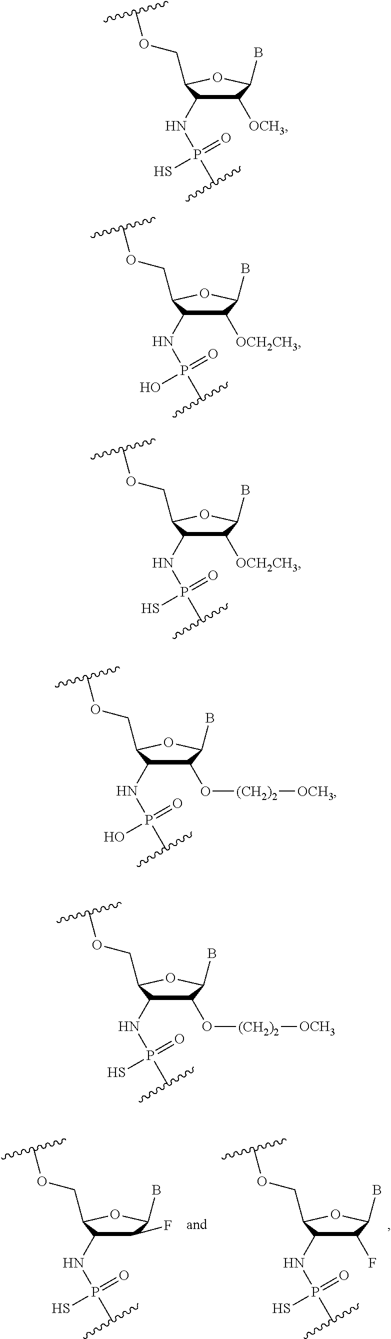

where B can be any natural or modified base.

Compounds of the present disclosure include compounds comprising the following Formula (V″):

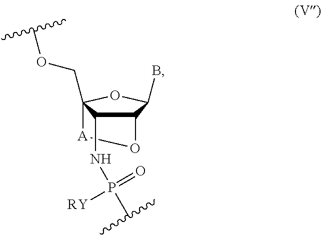

wherein Y is S or O, R is H or a positively charged counter ion, B is independently in each instance a natural or an unmodified nucleobase or a modified nucleobase, A is —(CR″R″)₁₋₂— and R″ is independently in each instance H, F or Me, and optionally comprising one or more of Formulae (I′), (II′), (III′), (IV′) or (V′).

In the compound comprising formula (V″), A is —(CR″R″)₁₋₂—. In some embodiments, A is —(CR″R″)— in other embodiments, A is —(CR″R″)₂—. R″ is independently in each instance H or Me. In some embodiments, one R″ is Me and remaining are H. In other embodiments, all R″ are H.

In some embodiments, when A is CH₂, then Y is S. In other embodiments, when A is CH₂CH₂, then Y is O or S. In some embodiments, A is CH₂CH(Me) or CH(Me) and Y is O or S.

In the compound comprising formula (V″), Y is O or S. In some embodiments, Y is S in at least one instance (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.). In other embodiments, Y is S in at least one instance and O in at least another instance. In other embodiments, Y is S in each instance. In some embodiments, Y is O in at least one instance (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 etc.).

The compound of Formula (V″) (and optionally Formulae (I′), (II′), (III′), (IV′), and/or (V′) may be part of an oligonucleotide. In some embodiments, the compound comprising Formula (IV′) (and optionally Formulae (I′), (II′), (III′), (IV′) and/or (V′) is an oligonucleotide comprising 1, 2, 3, 4, 5, 6, 7, 8, 9, 10, 11, 12, 13, 14, 15, 16, 17, 18, 19, 20, 21, 22, 23, 24 nucleotides of Formula (V″) (and Formulae (I′), (II′), (III′), (IV′) and/or (V′)). In some embodiments, the oligonucleotide comprises from 2 to 40 nucleotides, for example, 8 to 26 nucleotides or integers there between.

In embodiments where more than one nucleotides of Formula (V′) are included, the more than one nucleotides of Formula (V′) may be the same or different. In some embodiments one or more nucleotides of Formulae (I′), (II′), (III′), (IV′), and/or (V′) are included, and may be the same or different. For example, in some embodiments, the nucleotide comprises at least one nucleotide of Formula (V′) and at least one nucleotide of Formulae (I′), (II′), (III′), (IV′), and/or (V′). In some embodiments, the nucleotide comprises at least 2 alternating nucleotides of Formula (V″) and Formula (I′) and/or (II′). For example, 2, 3, 4, 5, 6, 7, 8, 9, 10, 11, 12, 13, 14, 15, 16, 17, 18, 19, 20, 21, 22, 23, 24 nucleotides with alternating 2′ modification.

In some embodiments, the nucleotide comprising the nucleotide of Formula (V′) (and optionally Formulae (I′), (II′), (III′), (IV′), and/or (V′)) further comprises a 2-fluoronucleotide of the following structures:

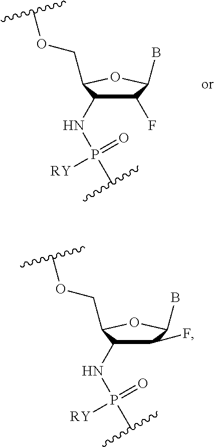

where Y, R and B are the same as for Formula (I′). In some embodiments, the nucleotide comprises at least 4 alternating nucleotides of Formula (V′) and 2-fluoronucleotides.

Compounds of the present disclosure include compounds comprising the following Formula (V′):

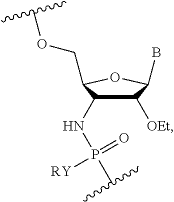

wherein Y is S or O, R is H or a positively charged counter ion, and B is independently in each instance a natural or an unmodified nucleobase or a modified nucleobase; and optionally comprising one or more of formula (I′), (II′), (III′), (IV′) , (V′) and/or (V″).

The following abbreviations are used in this disclosure. 2′-H (deoxyribose) nucleosides are referred to by an uppercase letter corresponding to the nucleobase, e.g., A, C, G and T. 2′-O-Methylated (2′-O-Me) nucleosides are referred to by a lowercase m and an uppercase letter corresponding to the nucleobase, e.g., mA, mC, mG and mU. 2′-O-Me, 5-methylcytosine is abbreviated as 5mmC.

For the backbone or intersubunit linkages of the nucleotides, phosphodiester intersubunit linkages are referred to as “PO” or are generally not included in sequence details; thiophosphate intersubunit linkages are abbreviated as lowercase “ps”; phosphoramidate intersubunit linkages are abbreviated as lowercase “np”; and thiophosphoramidate intersubunit linkages are abbreviated as lowercase “nps.”

In embodiments, at least one nucleotide of any one of SEQ ID NOs: 1-65 is modified to include a 5-methylcytosine nucleobase, an O-Me modification at the 2′ position (mA, 5mmC, mG and mU), and a phosphorothioate (PS) linkage between nucleotides. In embodiments, each nucleotide of any one of SEQ ID NOs: 1-65 are modified as follows:
(a) each nucleotide having a cytosine nucleobase is modified to be a 2′-O-Me, 5-methylcytosine (5mmC);
(b) each other nucleotide is also modified to include an O-Me modification at the 2′ position (mA, mG and mU); and
(c) each nucleotide contains a phosphorothioate (PS) linkage between nucleotides.

For example, the SEQ ID NOs: 1-13 can be modified as set forth in Table 2. An oligonucleotide of SEQ ID NO: 1 may be modified as SEQ ID NO: 66. An oligonucleotide of SEQ ID NO: 2 may be modified as SEQ ID NO: 67. An oligonucleotide of SEQ ID NO: 3 may be modified as SEQ ID NO: 68. An oligonucleotide of SEQ ID NO: 4 may be modified as SEQ ID NO: 69. An oligonucleotide of SEQ ID NO: 5 may be modified as SEQ ID NO: 70. An oligonucleotide of SEQ ID NO: 6 may be modified as SEQ ID NO: 71. An oligonucleotide of SEQ ID NO: 7 may be modified as SEQ ID NO: 72. An oligonucleotide of SEQ ID NO: 8 may be modified as SEQ ID NO: 74. An oligonucleotide of SEQ ID NO: 9 may be modified as SEQ ID NO: 75. An oligonucleotide of SEQ ID NO: 10 may be modified as SEQ ID NO: 76 An oligonucleotide of SEQ ID NO: 11 may be modified as SEQ ID NO: 77. An oligonucleotide of SEQ ID NO: 12 may be modified as SEQ ID NO: 78. An oligonucleotide of SEQ ID NO: 13 may be modified as SEQ ID NO: 79.

In embodiments, at least one nucleotide of any one of SEQ ID NOs: 1-65 is modified to include a 5-methylcytosine nucleobase, an O-Me modification at the 2′ position (mA, 5mmC, mG and mU), and a thiophosphoroamidate (NPS) linkage between nucleotides. In embodiments, each nucleotide of any one of SEQ ID NOs: 1-65 are modified as follows:
(a) each nucleotide having a cytosine nucleobase is modified to be a 2′-O-Me, 5-methylcytosine (5mmC);
(b) each other nucleotide is also modified to include an O-Me modification at the 2′ position (mA, mG and mU); and
(c) each nucleotide contains a thiophosphoroamidate (NPS) linkage between nucleotides.

For example, an oligonucleotide of SEQ ID NO: 7 may be modified as SEQ ID NO: 73 as set forth in Table 2.

TABLE 2
Modified Oligonucleotide Sequences (SEQ ID NO)

5′-mApsmApsmGps5mmCps5mmCps5mmCps5mmCpsmApsmGps5mm
Cps5mmCpsmApsmGpsmUpsmGpsmGpsmGpsmGpsmGpsmUpsmU-3′
(SEQ ID NO: 66)
5′-5mmCps5mmCpsmApsmApsmGps5mmCps5mmCps5mmCps5mm
CpsmApsmGps5mmCps5mmCpsmApsmGpsmUpsmGpsmGpsmG-3′
(SEQ ID NO: 67)
5′-5mmCpsmUpsmUpsmGpsmUpsmApsmApsmGpsmUpsmUpsmGpsm
Gps5mmCpsmGpsmApsmGpsmApsmApsmApsmG-3′ (SEQ ID NO:
68)
5′-mUpsmAps5mmCpsmUpsmUpsmUps5mmCps5mmCpsmApsmApsm
Ups5mmCpsmApsmApsmUpsmApsmGpsmGps5mmCps-3′ (SEQ ID
NO: 69)
5′-5mmCps5mmCpsmUpsmApsmUpsmUpsmGpsmApsmUpsmUpsm
GpsmGpsmApsmApsmApsmGpsmU-3′ (SEQ ID NO: 70)
5′-mGps5mmCps5mmCpsmUpsmApsmUpsmUpsmGpsmApsmUpsm
UpsmGpsmGpsmApsmApsmApsmGpsmUpsmA-3′ (SEQ ID NO:
71)
5′-mUpsmGpsmApsmAps5mmCps5mmCpsmUpsmUpsmUpsmAps5mm
Cps5mmCps5mmCps5mmCpsmGpsmUpsmUpsmGps5mmCps5mmC-3′
(SEQ ID NO: 72)
5′-mUnpsmGnpsmAnpsmAnps5mmCnps5mmCnpsmUnpsmUnpsm
UnpsmAnps5mmCnps5mmCnps5mmCnps5mmCnpsmGnpsmUnpsm
UnpsmGnps5mmCnps5mmC-3′ (SEQ ID NO: 73)
5′-mUpsmGps5mmCpsmGpsmUps5mmCpsmApsmGps5mmCpsmApsm
ApsmAps5mmCpsmAps5mmCpsmUpsmUpsmGpsmGps5mmCps-3′
(SEQ ID NO: 74)
5′-mGps5mmCpsmGpsmUps5mmCpsmApsmGps5mmCpsmApsmApsm
Aps5mmCpsmAps5mmCpsmUpsmUpsmGpsmGps5mmCpsmA-3′
(SEQ ID NO: 75)
5′-mUps5mmCpsmUps5mmCpsmGps5mmCps5mmCpsmApsmAps5mm
CpsmUpsmUpsmAps5mmCpsmApsmApsmGpsmGps5mmCps5mmC-3′
(SEQ ID NO: 76)
5′-mApsmUpsmUpsmGpsmApsmUpsmUpsmGpsmGpsmApsmApsm
ApsmGpsmU-3′ (SEQ ID NO: 77)
5′-mGps5mmCps5mmCpsmApsmApsmGpsmUpsmGpsmUpsmUpsm
UpsmGps5mmCpsmUpsmGpsmAps5mmCpsmGps5mmC-3′ (SEQ ID
NO: 78)
5′-mGps5mmCpsmUps5mmCpsmGps5mmCpsmApsmGps5mmCps5mm
CpsmGpsmGpsmUps5mmCpsmUpsmGpsmGpsmApsmG-3′ (SEQ ID
NO: 79)

In embodiments, a ligand-targeting moiety is conjugated to the oligonucleotide. Targeting moieties include GalNAc such as GalNAc-1-13. For example, the following GalNAc derivatives are included in some embodiments. The following show the GalNAc moiety attached to a linker or support, as indicated in the structure.

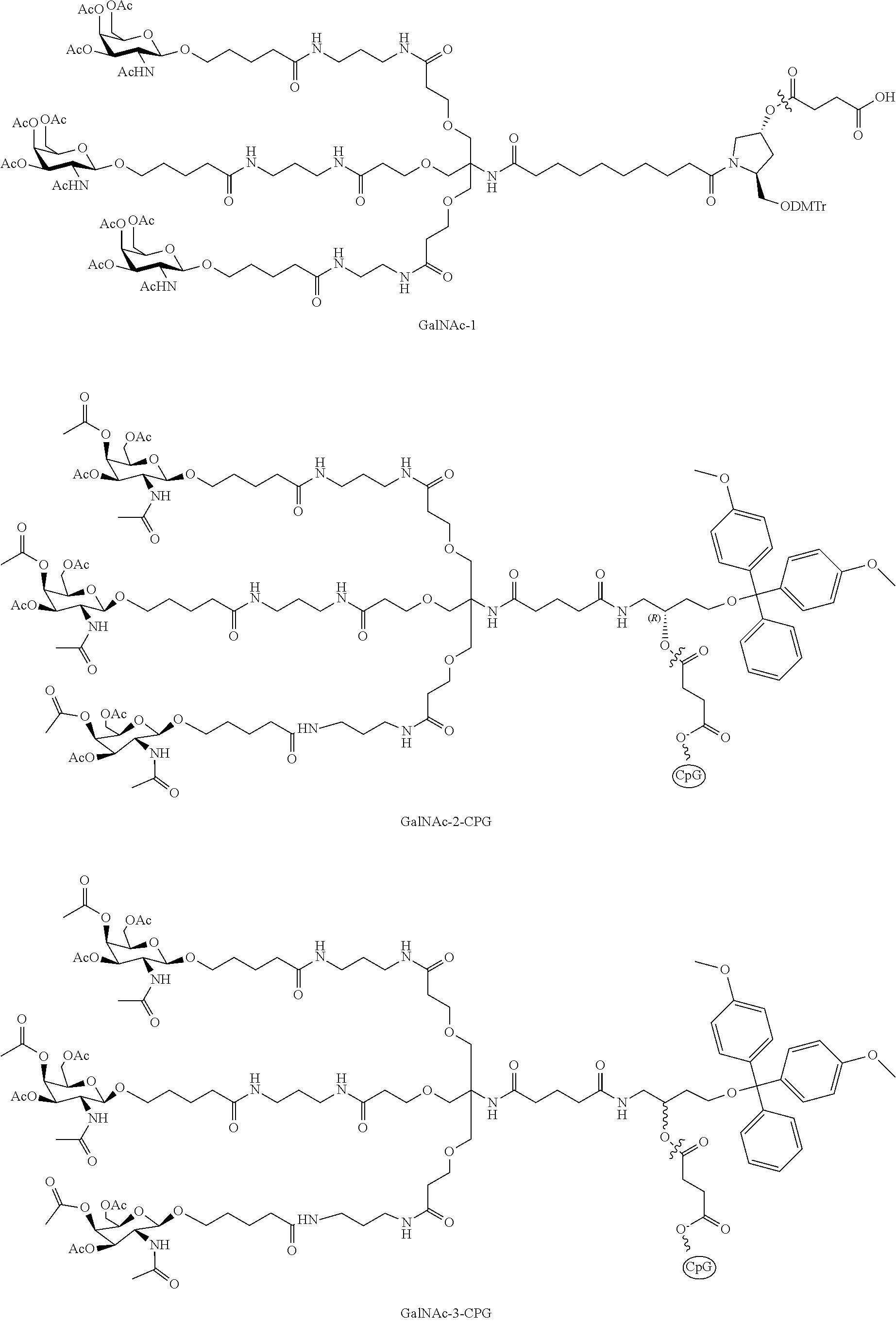

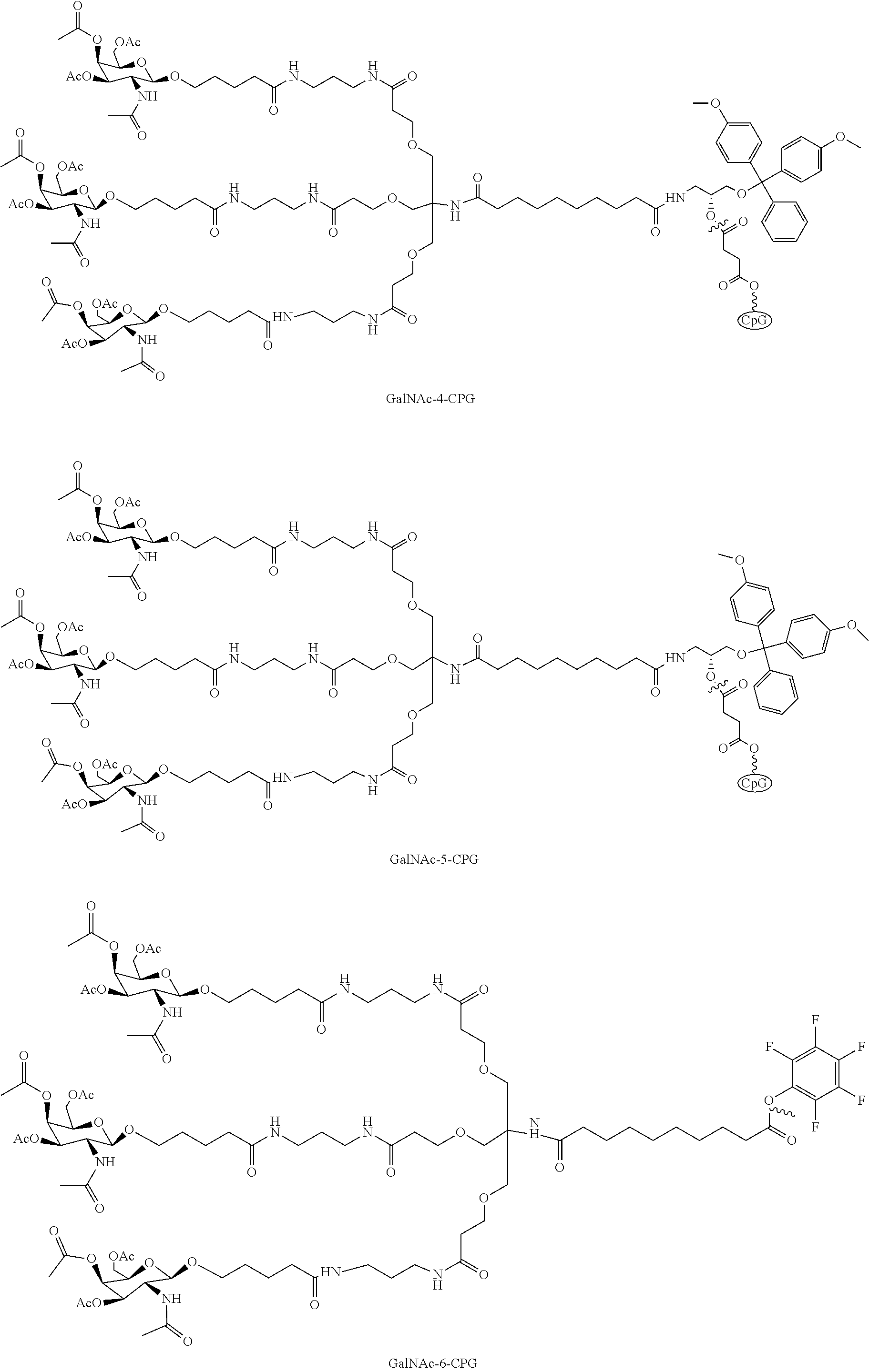

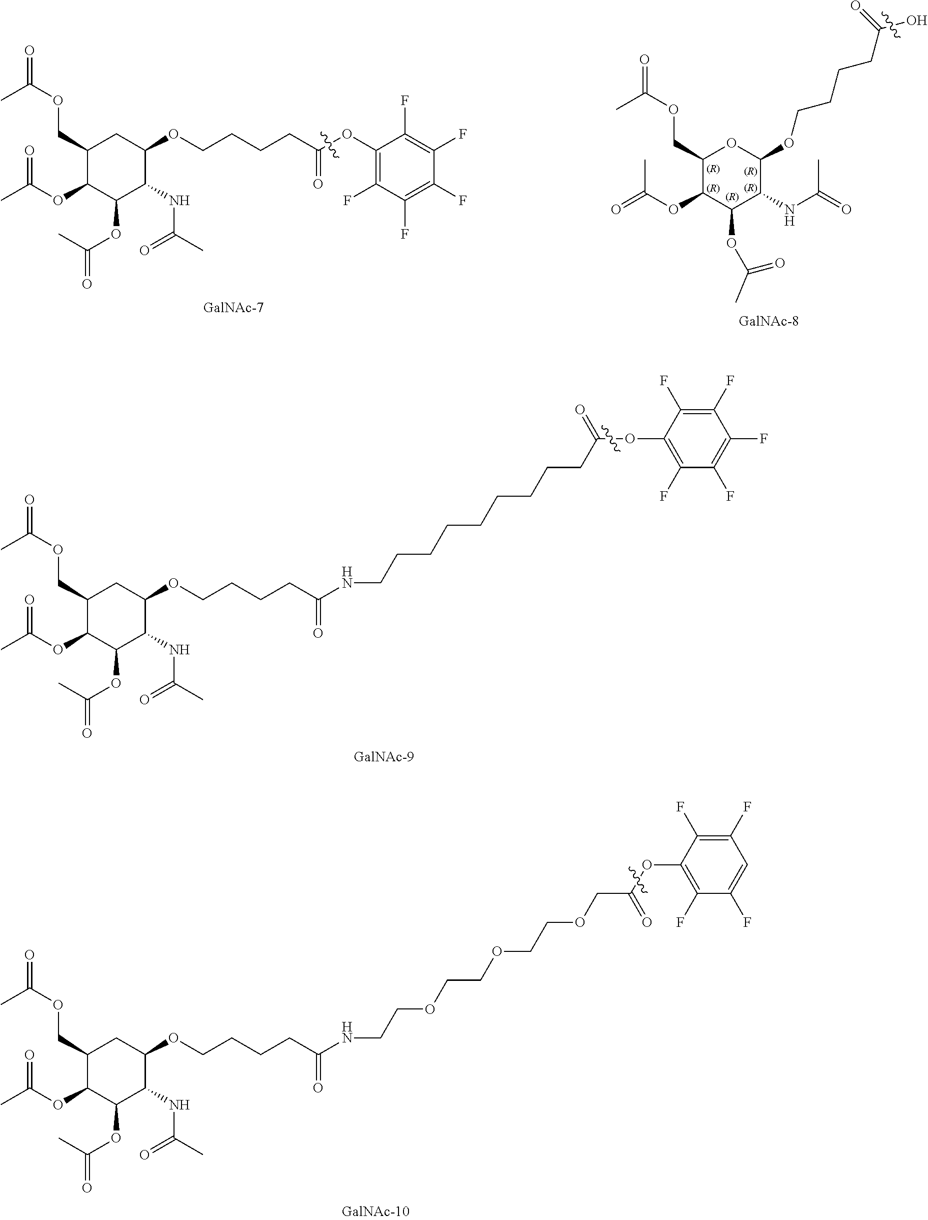

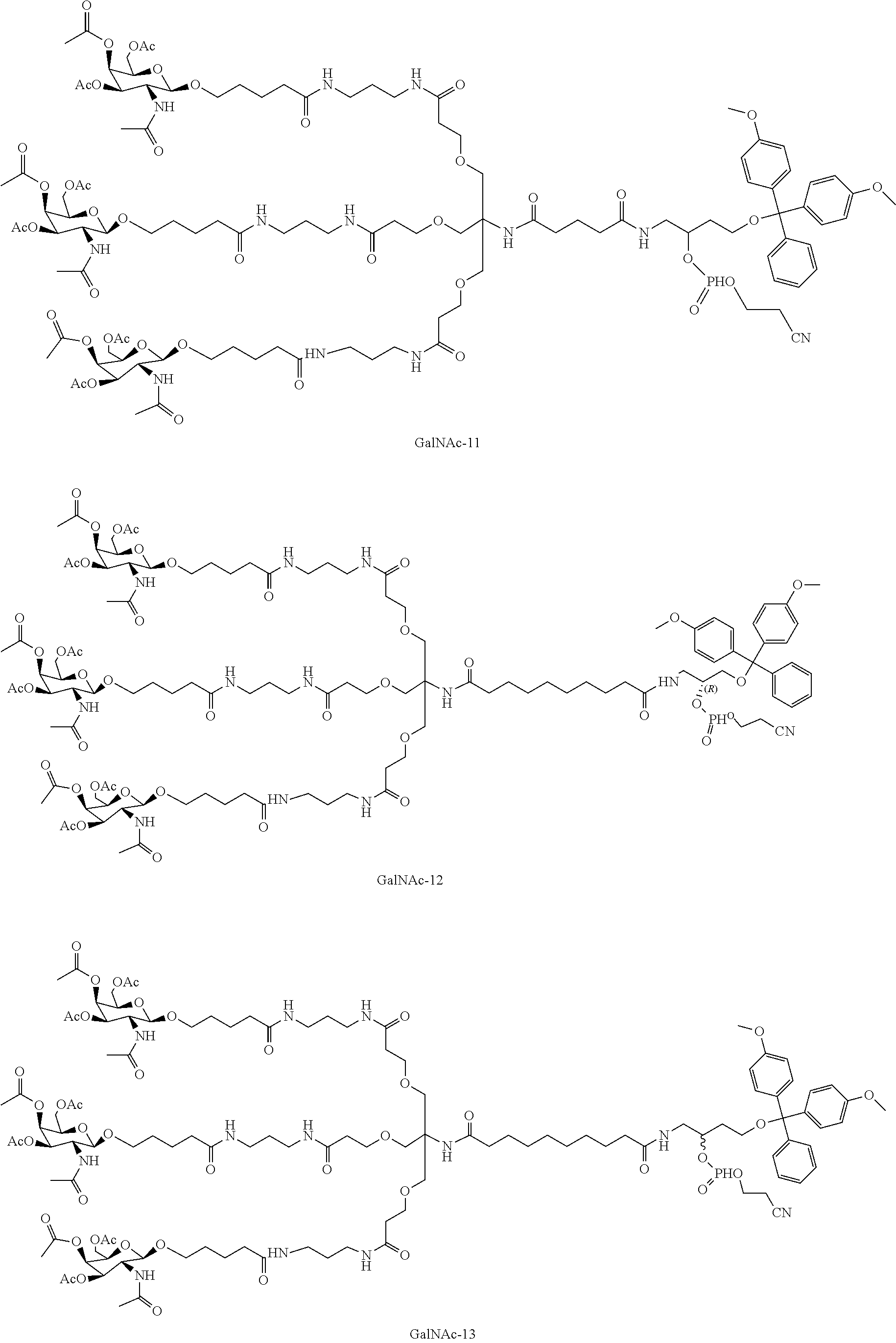

The GalNAc derivative may be conjugated at 3′ and/or 5′ end of the oligonucleotides of the present disclosure. For example, oligonucleotides of SEQ ID NO: 71 may include a 3′GalNAc (SEQ ID NO: 80). Other targeting moieties may include palmitoyl or tocopherol modifications. For example, oligonucleotides of SEQ ID NO: 71 may include a 3′ palmitoyl (SEQ ID NO: 81) or a 3′ tocopherol (SEQ ID NO: 82).

### Therapeutic and Prophylactic Methods

The following discussion is presented by way of example only, and is not intended to be limiting.

One aspect of the present disclosure includes methods for treating a subject diagnosed as having, suspected as having, or at risk of having an HBV infection and/or an HBV-associated disorder. In therapeutic applications, compositions comprising the oligonucleotides of the present disclosure are administered to a subject suspected of, or already suffering from such a disease (such as, e.g., persistence of HBV cccDNA, presence of an HBV antigen (e.g., HBsAg and/or HBeAg) in the serum and/or liver of the subject, or elevated HBV viral load levels), in an amount sufficient to cure, or at least partially arrest, the symptoms of the disease, including its complications and intermediate pathological phenotypes in development of the disease.

Subjects suffering from an HBV infection and/or an HBV-associated disorder can be identified by any or a combination of diagnostic or prognostic assays known in the art, including detection of typical symptoms of HBV infection and/or an HBV-associated disorder described herein.

The present disclosure provides a method for treating a subject diagnosed as having, or suspected as having an HBV infection and/or an HBV-associated disorder comprising administering to the subject an effective amount of an oligonucleotide composition of the present disclosure.

In some embodiments, subjects treated with the oligonucleotide composition of the present disclosure will show amelioration or elimination of one or more of the following symptoms: presence of liver HBV cccDNA, the presence of serum and/or liver HBV antigen (e.g., HBsAg and/or HBeAg), the absence or low level of anti-HBV antibodies, liver injury, cirrhosis, delta hepatitis, acute hepatitis B, acute fulminant hepatitis B, chronic hepatitis B, liver fibrosis, end-stage liver disease, hepatocellular carcinoma, serum sickness-like syndrome, anorexia, nausea, vomiting, low-grade fever, myalgia, fatigability, disordered gustatory acuity and smell sensations (aversion to food and cigarettes), right upper quadrant and epigastric pain (intermittent, mild to moderate), hepatic encephalopathy, somnolence, disturbances in sleep pattern, mental confusion, coma, ascites, gastrointestinal bleeding, coagulopathy, jaundice, hepatomegaly (mildly enlarged, soft liver), splenomegaly, palmar erythema, spider nevi, muscle wasting, spider angiomas, vasculitis, variceal bleeding, peripheral edema, gynecomastia, testicular atrophy, abdominal collateral veins (caput medusa), ALT levels higher than AST levels, leukopenia (i.e., granulocytopenia), decreased albumin levels, elevated serum iron levels, lymphocytosis, increased erythrocyte sedimentation rate (ESR), shortened red blood cell survival, hemolysis, thrombocytopenia, a prolongation of the international normalized ratio (INR), the presence of serum HBV DNA, prolonged prothrombin time (PT), hyperglobulinemia, the presence of tissue-nonspecific antibodies, such as anti-smooth muscle antibodies (ASMAs) or antinuclear antibodies (ANAs), the presence of tissue-specific antibodies, such as antibodies against the thyroid gland, hyperbilirubinemia, low platelet and white blood cell counts, AST levels higher than ALT levels, lobular inflammation accompanied by degenerative and regenerative hepatocellular changes, and predominantly centrilobular necrosis.

In some embodiments, subjects treated with the oligonucleotide composition of the present disclosure will show a reduction in the expression levels of one or more biomarkers selected from among alanine aminotransferase (ALT), aspartate aminotransferase (AST), gamma-glutamyl transpeptidase (GGT), alkaline phosphatase (ALP), bilirubin, and rheumatoid factor (RF), compared to untreated subjects suffering from an HBV infection and/or an HBV-associated disorder.

In some embodiments, an oligonucleotide that targets HBV cccDNA is administered to a subject having an HBV infection and/or an HBV-associated disease such that one or more of: HBV cccDNA levels, HBV antigen levels, HBV viral load levels, ALT levels, and/or AST levels, e.g., in a cell, tissue, blood or other biological fluid of the subject are reduced by at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% 60%, 61%, 62%, 63%, 64%, 65%, 66% 67%, 68%, 69%, 70%, 71% 72%, 73%, 74%, 75%, 76%, 77%, 78%, 79%, 80%, 81%, 82%, 83%, 84%, 85%, 86%, 7%, 88%, 89%, 90%, 91%, 92%, 93%, 94%, 95%, 96%, 97%, 98%, or at least about 99% or more compared to levels observed in the subject prior to the administration of the oligonucleotide.

In some embodiments, an oligonucleotide: that targets HBV cccDNA is administered to a subject having an HBV infection and/or an HBV-associated disease such that the level of anti-HBV antibodies, e.g., in a cell, tissue, blood or other biological fluid of the subject are increased by at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76% 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more compared to levels observed in the subject prior to the administration of the oligonucleotide.

The methods of the present disclosure include administering an oligonucleotide composition described herein such that HBV cccDNA levels are reduced for about 1, 2, 3, 4 5, 6, 7, 8, 12, 16, 18, 24, 28, 32, 36, 40, 44, 48, 52, 56, 60, 64, 68, 72, 76, or about 80 hours. In one embodiment, HBV cccDNA levels are decreased for an extended duration, e.g., at least about two, three, four, five, six, seven days or more, or about one week, two weeks, three weeks, or about four weeks or more.

In some embodiments, administration of the oligonucleotide compositions of the present disclosure reduces the presence of liver HBV cccDNA, the level of HBV DNA (e.g., rcDNA), the presence of serum and/or liver HBV antigens (e.g., FIBsAg and/or HBeAg), ALT levels, and/or AST levels, e.g., in a cell, tissue, blood, urine or organ of the patient by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% 39%, 40%, 41%, 42%, 43%, 44% 45% 46%, 47% 48%, 49%, 50%, 51%, 52%, 53% 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more, e.g., to below the level of detection of the assay.

Additionally or alternatively, in some embodiments, administration of the oligonucleoti de compositions of the present disclosure increases the presence of serum and/or liver anti-HBV antibodies, e.g., in a cell, tissue, blood, urine or organ of the patient by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90% 92%, 93%, 94% 95%, 96%, 97%, 98%, or at least about 99% or more.

In one aspect, the present disclosure provides a method for inducing D-loop formation in HBV cccDNA comprising contacting HBV cccDNA with an oligonucleotide having a sequence of any one of SEQ ID NOs: 1-82. In another aspect, the present disclosure provides a method for inducing D-loop formation in HBV cccDNA comprising contacting a target region of an HBV cccDNA genome consisting of nucleotide position 900-1310 (Enhancer I region) with an oligonucleotide that is at least 90% complementary to the target region of the HBV cccDNA. In some embodiments, the oligonucleotides disclosed herein hybridize with HBV cccDNA to induce the formation of an antigenic D-loop structure. In some embodiments, induction of D-loop formation stimulates innate immunity.

For therapeutic applications, an oligonucleotide composition of the present disclosure is administered to the subject. In some embodiments, the oligonucleotide composition is administered one, two, three, four, or five times per day. In some embodiments, the oligonucleotide composition is administered more than five times per day. Additionally or alternatively, in some embodiments, the oligonucleotide composition is administered every day, every other day, every third day, every fourth day, every fifth day, or every sixth day. In some embodiments, the oligonucleotide composition is administered weekly, bi-weekly, tri-weekly, or monthly. In some embodiments, the oligonucleotide composition is administered for a period of one, two, three, four, or five weeks. In some embodiments, the oligonucleotide composition is administered for six weeks or more. In some embodiments, the oligonucleotide composition is administered for twelve weeks or more. In some embodiments, the oligonucleotide composition is administered for a period of less than one year. In some embodiments, the oligonucleotide composition is administered for a period of more than one year.

In some embodiments of the methods of the present disclosure, the oligonucleotide composition is administered daily for 1 week or more. In some embodiments of the methods of the present disclosure, the oligonucleotide composition is administered daily for 2 weeks or more. In some embodiments of the methods of the present disclosure, the oligonucleotide composition is administered daily for 3 weeks or more. In some embodiments of the methods of the present disclosure, the oligonucleotide composition is administered daily for 4 weeks or more. In some embodiments of the methods of the present disclosure, the oligonucleotide composition is administered daily for 6 weeks or more. In some embodiments of the methods of the present disclosure, the oligonucleotide composition is administered daily for 12 weeks or more.

Efficacy of treatment of the disease can be assessed, for example by measuring disease progression, disease remission, symptom severity, reduction in pain, quality of life, dose of a medication required to sustain a treatment effect, level of a disease marker or any other measurable parameter appropriate for a given disease being treated. It is well within the ability of one skilled in the art to monitor efficacy of treatment by measuring any one of such parameters, or any combination of parameters. For example, efficacy of treatment of CHB may be assessed, for example, by periodic monitoring of viral load and transaminase levels. Comparison of the later readings with the initial readings provides an indication of whether the treatment is effective.

### Modes of Administration

The present disclosure provides pharmaceutical compositions and formulations comprising the oligonucleotides of the present disclosure. The pharmaceutical compositions are useful for treating a disease or disorder associated with HBV infection.

In certain embodiments, the pharmaceutical compositions include an oligonucleotide, as described herein, and a pharmaceutically acceptable carrier. Such pharmaceutical compositions are formulated based on the mode of delivery.

The pharmaceutical compositions of the present disclosure can be administered in a number of ways depending upon whether local or systemic treatment is desired and upon the area to be treated. Administration can be topical, intrahepatic, transdermal (e.g., by a transdermal patch), pulmonary, e.g., by inhalation or insufflation of powders or aerosols, including by nebulizer, intratracheal, intranasal, epidermal, oral, rectal, or parenteral. Parenteral administration includes intravenous, intraarterial, subcutaneous, intraperitoneal or intramuscular injection or infusion, subdermal, e.g., via an implanted device, or intracranial, e.g., by intraparenchymal, intrathecal or intraventricular, administration.

In some embodiments, the pharmaceutical compositions are formulated for systemic administration via parenteral delivery, e.g., intravenously, intraarterially, intramuscularly, intraperitoneally, subdermally, intracranially, or subcutaneously. In certain embodiments, the pharmaceutical compositions are formulated for direct delivery into the brain parenchyma, e.g., infusion into the brain by continuous pump infusion.

In some embodiments, the administration is via a depot injection. A depot injection may release the oligonucleotide in a consistent way over a prolonged time period. Thus, a depot injection may reduce the frequency of dosing needed to obtain a desired therapeutic or prophylactic effect. A depot injection may also provide more consistent serum concentrations. Depot injections may include subcutaneous injections or intramuscular injections.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions. In certain embodiments, the infusion pump is a subcutaneous infusion pump. In other embodiments, the pump is a surgically implanted pump that delivers the oligonucleotide to the liver.

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be included. Suitable topical formulations include those in which the oligonucleotides featured in the present disclosure are in admixture with a topical delivery agent such as lipids, liposomes, fatty acids, fatty acid esters, steroids, chelating agents and surfactants. Suitable lipids and liposomes may be neutral (e.g., dioleoylphosphatidyl DOPE ethanolamine, dimyristoylphosphatidyl choline DMPC, di stearolyphosphatidyl choline), anionic (e.g., dimyristoylphosphatidyl glycerol DMPG), or cationic (e.g., dioleoyltetramethylaminopropyl DOTAP and dioleoylphosphatidyl ethanolamine DOTMA). Oligonucleotides featured in the present disclosure can be encapsulated within liposomes or can form complexes thereto, in particular to cationic liposomes. Alternatively, oligonucleotides can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

For oral administration, liquid or solid formulations may be used. Examples of such formulations include tablets, gelatin capsules, pills, troches, elixirs, suspensions, syrups, wafers, chewing gum and the like. The oligonucleotides of the present disclosure can be mixed with a suitable pharmaceutical carrier (vehicle) or excipient as understood by practitioners in the art. Examples of carriers and excipients include starch, milk, sugar, certain types of clay, gelatin, lactic acid, stearic acid or salts thereof, including magnesium or calcium stearate, talc, vegetable fats or oils, gums and glycols.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials may be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The pharmaceutical compositions of the present disclosure may be administered in dosages sufficient to target the cccDNA of HBV.

In one embodiment, an oligonucleotide of the present disclosure is administered to a subject as a weight-based dose. A “weight-based dose” (e.g., a dose in mg/kg) is a dose of the oligonucleotide that changes depending on the subject's weight. In another embodiment, an oligonucleotide is administered to a subject as a fixed dose. A “fixed dose” (e.g., a dose in mg) means that the same oligonucleotide dose is used for all subjects regardless of any specific subject-related factors, such as weight. In one particular embodiment, a fixed dose of an oligonucleotide of the present disclosure is based on a predetermined weight or age.

In general, a suitable dose of an oligonucleotide of the present disclosure will be in the range of about 0.0001 to about 200.0 milligrams per kilogram body weight of the recipient per day, or in the range of about 1 to 50 mg per kilogram body weight per day. For example, the oligonucleotide can be administered at about 0.01 mg/kg, about 0.05 mg/kg, about 0.5 mg/kg, about 1 mg/kg, about 1.5 mg/kg, about 2 mg/kg, about 3 mg/kg, about 10 mg/kg, about 20 mg/kg, about 30 mg/kg, about 40 mg/kg, or about 50 mg/kg per day.

Subjects can be administered a therapeutically effective amount of an oligonucleotide of the present disclosure, such as about 0.01 mg/kg, 0.02 mg/kg, 0.03 mg/kg, 0.04 mg/kg, 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 15 mg/kg, 20 mg/kg, 25 mg/kg, 30 mg/kg, 35 mg/kg, 40 mg/kg, 45 mg/kg, or about 50 mg/kg oligonucleotide. Values and ranges intermediate to the recited values are also contemplated as part of the present disclosure.

In some embodiments, the oligonucleotide may be administered at a dose of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or about 10 mg/kg. Values and ranges intermediate to the recited values are also contemplated as part of the present disclosure.

In another embodiment, the oligonucleotide is administered at a dose of about 0.1 to about 50 mg/kg, about 0.25 to about 50 mg/kg, about 0.5 to about 50 mg/kg, about 0.75 to about 50 mg/kg, about 1 to about 50 mg/kg, about 1.5 to about 50 mg/kg, about 2 to about 50 mg/kg, about 2.5 to about 50 mg/kg, about 3 to about 50 mg/kg, about 3.5 to about 50 mg/kg, about 4 to about 50 mg/kg, about 4.5 to about 50 mg/kg, about 5 to about 50 mg/kg, about 7.5 to about 50 mg/kg, about 10 to about 50 mg/kg, about 15 to about 50 mg/kg, about 20 to about 50 mg/kg, about 25 to about 50 mg/kg, about 30 to about 50 mg/kg, about 35 to about 50 mg/kg, about 40 to about 50 mg/kg, about 45 to about 50 mg/kg, about 0.1 to about 45 mg/kg, about 0.25 to about 45 mg/kg, about 0.5 to about 45 mg/kg, about 0.75 to about 45 mg/kg, about 1 to about 45 mg/kg, about 1.5 to about 45 mg/kg, about 2 to about 45 mg/kg, about 2.5 to about 45 mg/kg, about 3 to about 45 mg/kg, about 3.5 to about 45 mg/kg, about 4 to about 45 mg/kg, about 4.5 to about 45 mg/kg, about 5 to about 45 mg/kg, about 7.5 to about 45 mg/kg, about 10 to about 45 mg/kg, about 15 to about 45 mg/kg, about 20 to about 45 mg/kg, about 25 to about 45 mg/kg, about 30 to about 45 mg/kg, about 35 to about 45 mg/kg, about 40 to about 45 mg/kg, about 0.1 to about 40 mg/kg, about 0.25 to about 40 mg/kg, about 0.5 to about 40 mg/kg, about 0.75 to about 40 mg/kg, about 1 to about 40 mg/kg, about 1.5 to about 40 mg/kg, about 2 to about 40 mg/kg, about 2.5 to about 40 mg/kg, about 3 to about 40 mg/kg, about 3.5 to about 40 mg/kg, about 4 to about 40 mg/kg, about 4.5 to about 40 mg/kg, about 5 to about 40 mg/kg, about 7.5 to about 40 mg/kg, about 10 to about 40 mg/kg, about 15 to about 40 mg/kg, about 20 to about 40 mg/kg, about 25 to about 40 mg/kg, about 30 to about 40 mg/kg, about 35 to about 40 mg/kg, about 0.1 to about 30 mg/kg, about 0.25 to about 30 mg/kg, about 0.5 to about 30 mg/kg, about 0.75 to about 30 mg/kg, about 1 to about 30 mg/kg, about 1.5 to about 30 mg/kg, about 2 to about 30 mg/kg, about 2.5 to about 30 mg/kg, about 3 to about 30 mg/kg, about 3.5 to about 30 mg/kg, about 4 to about 30 mg/kg, about 4.5 to about 30 mg/kg, about 5 to about 30 mg/kg, about 7.5 to about 30 mg/kg, about 10 to about 30 mg/kg, about 15 to about 30 mg/kg, about 20 to about 30 mg/kg, about 25 to about 30 mg/kg, about 0.1 to about 20 mg/kg, about 0.25 to about 20 mg/kg, about 0.5 to about 20 mg/kg, about 0.75 to about 20 mg/kg, about 1 to about 20 mg/kg, about 1.5 to about 20 mg/kg, about 2 to about 20 mg/kg, about 2.5 to about 20 mg/kg, about 3 to about 20 mg/kg, about 3.5 to about 20 mg/kg, about 4 to about 20 mg/kg, about 4.5 to about 20 mg/kg, about 5 to about 20 mg/kg, about 7.5 to about 20 mg/kg, about 10 to about 20 mg/kg, or about 15 to about 20 mg/kg. Values and ranges intermediate to the recited values are also contemplated as part of the present disclosure.

In some embodiments, the oligonucleotide may be administered at a dose of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or about 10 mg/kg. Values and ranges intermediate to the recited values are also contemplated as part of the present disclosure.

In certain embodiments, subjects can be administered a single therapeutically effective amount of oligonucleotide, such as about 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, or about 50 mg/kg. Values and ranges intermediate to the recited values are also contemplated as part of the present disclosure.

In other embodiments, subjects are administered multiple doses of a therapeutically effective amount of oligonucleotide, such as a dose about 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, or about 50 mg/kg. A multi-dose regimen may include administration of a therapeutically effective amount of oligonucleotide daily, such as for two days, three days, four days, five days, six days, seven days, or longer.

In other embodiments, subjects are administered a repeat dose of a therapeutically effective amount of oligonucleotide, such as a dose about 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, 0.975, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 35, 36, 37, 38, 39, 40, 41, 42, 43, 44, 45, 46, 47, 48, 49, or about 50 mg/kg. A repeat-dose regimen may include administration of a therapeutically effective amount of oligonucleotide on a regular basis, such as every other day, every third day, every fourth day, twice a week, once a week, every other week, or once a month.

For example, the oligonucleotide of the present disclosure, e.g., an oligonucleotide in a pharmaceutical composition, may be administered at a dose of about 0.01 mg/kg, 0.0125 mg/kg, 0.015 mg/kg, 0.0175 mg/kg, 0.02 mg/kg, 0.0225 mg/kg, 0.025 mg/kg, 0.0275 mg/kg, 0.03 mg/kg, 0.0325 mg/kg, 0.035 mg/kg, 0.0375 mg/kg, 0.04 mg/kg, 0.0425 mg/kg, 0.045 mg/kg, 0.0475 mg/kg, 0.05 mg/kg, 0.0525 mg/kg, 0.055 mg/kg, 0.0575 mg/kg, 0.06 mg/kg, 0.0625 mg/kg, 0.065 mg/kg, 0.0675 mg/kg, 0.07 mg/kg, 0.0725 mg/kg, 0.075 mg/kg, 0.0775 mg/kg, 0.08 mg/kg, 0.0825 mg/kg, 0.085 mg/kg, 0.0875 mg/kg, 0.09 mg/kg, 0.0925 mg/kg, 0.095 mg/kg, 0.0975 mg/kg, 0.1 mg/kg, 0.125 mg/kg, 0.15 mg/kg, 0.175 mg/kg, 0.2 mg/kg, 0.225 mg/kg, 0.25 mg/kg, 0.275 mg/kg, 0.3 mg/kg, 0.325 mg/kg, 0.35 mg/kg, 0.375 mg/kg, 0.4 mg/kg, 0.425 mg/kg, 0.45 mg/kg, 0.475 mg/kg, or about 0.5 mg/kg. Values intermediate to the foregoing recited values are also intended to be part of the present disclosure.

In some embodiments, the oligonucleotide is administered as a fixed dose of between about 100 mg to about 900 mg, between about 100 mg to about 850 mg, between about 100 mg to about 800 mg, between about 100 mg to about 750 mg, between about 100 mg to about 700 mg, between about 100 mg to about 650 mg, between about 100 mg to about 600 mg, between about 100 mg to about 550 mg, between about 100 mg to about 500 mg, between about 200 mg to about 850 mg, between about 200 mg to about 800 mg, between about 200 mg to about 750 mg, between about 200 mg to about 700 mg, between about 200 mg to about 650 mg, between about 200 mg to about 600 mg, between about 200 mg to about 550 mg, between about 200 mg to about 500 mg, between about 300 mg to about 850 mg, between about 300 mg to about 800 mg, between about 300 mg to about 750 mg, between about 300 mg to about 700 mg, between about 300 mg to about 650 mg, between about 300 mg to about 600 mg, between about 300 mg to about 550 mg, between about 300 mg to about 500 mg, between about 400 mg to about 850 mg, between about 400 mg to about 800 mg, between about 400 mg to about 750 mg, between about 400 mg to about 700 mg, between about 400 mg to about 650 mg, between about 400 mg to about 600 mg, between about 400 mg to about 550 mg, or between about 400 mg to about 500 mg.

In some embodiments, the oligonucleotide is administered as a fixed dose of about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, or about 900 mg.

The pharmaceutical composition can be administered by intravenous infusion over a period of time, such as over a 5, 6, 7, 8, 9, 10, 11, 12, 13, 14, 15, 16, 17, 18, 19, 20, 21, 22, 23, 24, or about a 25 minute period. The administration may be repeated, for example, on a regular basis, such as weekly, biweekly (i.e., every two weeks) for one month, two months, three months, four months or longer. After an initial treatment regimen, the treatments can be administered on a less frequent basis. For example, after administration weekly or biweekly for three months, administration can be repeated once per month, for six months or a year or longer.

The pharmaceutical composition can be administered once daily, or the oligonucleotide can be administered as two, three, or more sub-doses at appropriate intervals throughout the day or even using continuous infusion or delivery through a controlled release formulation. In one embodiment, the oligonucleotide contained in each sub-dose must be correspondingly smaller in order to achieve the total daily dosage. The dosage unit can also be compounded for delivery over several days, e.g., using a conventional sustained release formulation which provides sustained release of the oligonucleotide over a period of several days. Sustained release formulations are well known in the art and are particularly useful for delivery of agents at a particular site. In one embodiment, the dosage unit contains a corresponding multiple of the daily dose.

In other embodiments, a single dose of the pharmaceutical compositions can be long lasting, such that subsequent doses are administered at not more than 3, 4, or 5 day intervals, or at not more than 1, 2, 3, or 4 week intervals. In some embodiments, a single dose of the pharmaceutical compositions of the present disclosure is administered once per week. In other embodiments, a single dose of the pharmaceutical compositions of the present disclosure is administered bi-monthly. In some embodiments, a single dose of the pharmaceutical compositions of the present disclosure is administered once per month, once every other month, or once quarterly (i.e., every three months).

Estimates of effective dosages and in vivo half-lives for the individual oligonucleotides encompassed by the present disclosure can be made using conventional methodologies or on the basis of in vivo testing using an appropriate animal model.

In some embodiments of the method, the oligonucleotide is administered orally, topically, systemically, intravenously, subcutaneously, transdermally, intrathecally, intranasally, intraperitoneally, intrahepatically, or intramuscularly.

In some embodiments of the method, the oligonucleotide is administered daily for 1 week or more. In other embodiments of the method, the oligonucleotide is administered daily for 2 weeks or more. In certain embodiments of the method, the oligonucleotide is administered daily for 3 weeks or more. In some embodiments of the method, the oligonucleotide is administered daily for 4 weeks or more. In other embodiments of the method, the oligonucleotide is administered daily for 6 weeks or more. In some embodiments of the method, the oligonucleotide is administered daily for 12 weeks or more.

### Combination Therapies

In some embodiments, the oligonucleotide compositions of the present disclosure, may be combined with one or more additional therapeutic agents for the amelioration or treatment of an HBV infection or and/or an HBV-associated disorder. In combination therapies, it is understood that the oligonucleotide compositions of the present disclosure and one or more additional treatments for HBV infection may be administered simultaneously in the same or separate compositions, or administered separately, at the same time or sequentially.

Examples of additional therapeutic agents that can be used in combination therapy include, but are not limited to, an antiviral agent, a nucleotide analog, a nucleoside analog, a reverse transcriptase inhibitor (e.g., Tenofovir disoproxil fumarate (TDF), Tenofovir alafenamide, Lamivudine, Adefovir dipivoxil, Entecavir (ETV), Telbivudine, AGX-1009, emtricitabine, clevudine, ritonavir, dipivoxil, lobucavir, famvir, FTC, N-Acetyl-Cysteine (NAC), PC1323, theradigm-HBV, thymosin-alpha, CMX157, AGX-1009, and ganciclovir), an immune stimulator (e.g., pegylated interferon α-2a (PEG-IFN-Cc2a), Interferon α-2b, a recombinant human interleukin-7, and a Toll-like receptor 7 (TLR7) agonist), a therapeutic vaccine (e.g., GS-4774, DV-601, TG1050, Heplislav, ABX203, and INO-1800), a viral entry inhibitor (e.g., Myrcludex), siRNA (e.g., ARC520, ARC521, ALN-HBV, ARB-1467 etc.), an antisense oligonucleotide (e.g., IONIS-HBV-L Rx etc.), an oligonucleotide that inhibits the secretion or release of HBsAg (e.g., REP 9AC), a capsid inhibitor (e.g., Bay41-4109, NVR-1221, NVR 3-778, JNJ-379, AB-423, GLS-4, HAP-1, and AT-1), a cccDNA inhibitor (e.g., IHVR-25), TLR agonists (e.g., GS-9620, ARB-1598, ANA975, RG7795(ANA773), MEDI9197, PF-3512676, and IMO-2055), miRNA mimics or inhibitors, aptamers, steric blockers, short-activating RNA (saRNA), immunomodulatory oligonucleotides, or other therapeutic agents and/or procedures, e.g., liver transplant, and chemotherapy.

The multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

In some embodiments, in addition to the administration of the oligonucleotide, the method further comprises separately, sequentially or simultaneously administering to the subject one or more additional therapeutic agents selected from the group consisting of: an antiviral agent, a nucleotide analog, a nucleoside analog, a reverse transcriptase inhibitor, an immune stimulator, a therapeutic vaccine, a viral entry inhibitor, a capsid inhibitor, and a cccDNA inhibitor. In some embodiments, the reverse transcriptase inhibitor is Tenofovir disoproxil fumarate (TDF), Tenofovir alafenamide, Lamivudine, Adefovir dipivoxil, Entecavir (ETV), Telbivudine, AGX-1009, emtricitabine, clevudine, ritonavir, dipivoxil, lobucavir, famvir, FTC, N-Acetyl-Cysteine (NAC), PC1323, theradigm-HBV, thymosin-alpha, CMX157, AGX-1009, or ganciclovir.

In certain embodiments, the immune stimulator is pegylated interferon α-2a (PEG-IFN-Cc2a), Interferon α-2b, a recombinant human interleukin-7, or a Toll-like receptor 7 (TLR7) agonist. In some embodiments, the therapeutic vaccine is GS-4774, DV-601, or TG1050. In other embodiments, the viral entry inhibitor is Myrcludex and the cccDNA inhibitor is IHVR-25. In some embodiments, the capsid inhibitor is Bay41-4109, NVR-1221, NVR 3-778, or JNJ-379. In some embodiments, the siRNA is ARC520, ARC521, ALN-HBV, or ARB-1467, and the antisense oligonucleotide is IONIS-HBV-L Rx.

### Kits

The present disclosure also provides kits that target the cccDNA of hepatitis B virus (HBV). Kits of the present disclosure comprise one or more oligonucleotides comprising a sequence selected from the group consisting of SEQ ID NOs: 1-82, or modifications thereof, The kit may also comprise instructions for use, packages such as packaging intended for commercial sale and the like.

### EXAMPLES

### Example 1

### Oligonucleotides of the Present Disclosure Reduce Viral Antigens in Infected Primary Human Hepatocytes

All oligonucleotides were prepared by solid phase synthesis. About 200 different oligonucleotides were synthesized that span either the (+) or (−) DNA strands. The biological effects of the different oligonucleotides on HBV gene expression (e.g., HBsAg, HBeAg, tox) were screened in HBV (+) Primary Human Hepatocytes (PHH) at 3 different doses (0.3, 3, or 30 μM).

Culture and Infection. Primary human hepatocytes (PHH) (BioreclamationlVT, Baltimore, Md.) were thawed and plated on Biocoat™ Collagen I Cellware 96-well, 24-well, or 6-well plates (Corning Inc., Corning, N.Y.) according to the manufacturer's instructions. Cells were thawed in a water bath at 37° C. and spun at 100×g for 10 min in Primary Hepatocytes Thawing and Plating Medium (ThermoFisher Scientific, Inc., Waltham, Mass.). Cells were resuspended and plated in hepatocyte maintenance media (HMM): DMEM supplemented with FBS, Insulin, EGF and Dexamethasone (ThermoFisher Scientific, Inc., Waltham, Mass.) at 8×10⁵/ml in plate format. PHH were infected with 50 GE HBV 16 hrs after seeding in hepatocyte maintenance media (HMM) in the presence of 4% (wt/vol) PEG 8000 (Sigma). After 24 hours, virus containing media was removed, the cells washed four times, and further incubated in HMM for 4 days. After the 4-day period, PHH were treated with 0.0015-10 μM of an oligonucleotide or vehicle via transfection with Lipofactamine RNAiMax (ThermoFisher) according to the manufacturer's instruction. The treated PHH were then further incubated for 6 days with a single media change at the mid-point of the 6 day incubation period.

ELISA. HBeAg ELISA and HBsAg ELISA were each performed on the PHH supernatant with HBe ELISA kit (Autobio Diagnostics Co. Ltd., Zhengzhou, China) and sAg ELISA kit (Autobio Diagnostics Co. Ltd., Zhengzhou, China), respectively, according to the manufacturer's instructions.

qPCR. Total HBV DNA was extracted from PHH using the MagMax Total Nucleic Acid Isolation kit (ThermoFisher Scientific, Waltham, Mass.) according to the manufacturer's instructions, and quantified using a standard qPCR assay amplifying for the core region of HBV DNA.

Results. Of the 200 oligonucleotides tested in the PHH assay, oligonucleotides targeting sequences around the Enhancer I region of the HBV cccDNA genome, starting at around nucleotide position 960 and ending at around nucleotide position 1330, surprisingly showed significant antiviral activity based on reduction of HBeAg and HBsAg remaining in the supernatant of PHH treated with the disclosed oligonucleotides. Table 3 sets forth the oligonucleotide concentration at any one of the three doses tested: 0.3, 3.0 and 30 μM at which this antiviral activity was observed.

TABLE 3

| SEQ | | |
|---|---|---|
| ID | Nucleobase Sequence | Dose at which Antiviral |
| NO: | (5′-3′) | Activity observed, μM |
| 1 | AAGCCCCAGCCAGUGGGGGUU | 0.3 |
| 2 | CCAAGCCCCAGCCAGUGGG | 0.3 |
| 3 | CUUGUAAGUUGGCGAGAAAG | >30 |
| 4 | UACUUUCCAAUCAAUAGGC | >30 |
| 5 | CCUAUUGAUUGGAAAGU | 0.3 |
| 6 | GCCUAUUGAUUGGAAAGUA | 0.3 |
| 7 | UGAACCUUUACCCCGUUGCC | 0.3 |
| 8 | UGCGUCAGCAAACACUUGGC | 0.3 |
| 9 | GCGUCAGCAAACACUUGGCA | 0.3 |
| 10 | UCUCGCCAACUUACAAGGCC | 0.3 |
| 11 | AUUGAUUGGAAAGU | 3.0 |
| 12 | GCCAAGUGUUUGCUGACGC | >30 |
| 13 | GCUCGCAGCCGGUCUGGAG | 0.3 |
| 14 | ACUUUCCAAUCAAU | 30 |
| 15 | GGCAACGGGGUAAAGGUUCA | 30 |
| 16 | GCCGGGCAACGGGGUAAAGG | 30 |
| 17 | GCGUCAGCAAACACUUGGC | 0.3 |
| 18 | CCACGCAUGCGCUGAUGGCC | 0.3 |
| 19 | CCAGCCAGUGGGGGUUGCGUC | 0.3 |
| 20 | GCCCCCAGCCAGUGGGGGUU | 0.3 |
| 21 | AAGCCCCAGCCAGUGGGGG | 0.3 |
| 22 | AGCCAGUGGGGGUUGCGUC | 30 |
| 23 | UUCCACGCAUGCGCUGAUGG | 0.3 |
| 24 | AAAGGUUCCACGCAUGCGCA | 0.3 |
| 25 | UUCCGCAGUAUGGAUCGGC | 0.3 |
| 26 | CGCAGUAUGGAUCGGCAGAGG | 0.3 |
| 27 | AGGAGUUCCGCAGUAUGGAUC | 0.3 |
| 28 | GGCUGCGAGCAAAACAAGC | 0.3 |
| 29 | CCGGCUGCGAGCAAAACAAGC | 0.3 |
| 30 | CCGGCUGCGAGCAAAACAA | 0.3 |
| 31 | CCAGACCGGCUGCGAGCAAAA | 0.3 |
| 32 | CUCCAGACCGGCUGCGAGC | 0.3 |
| 33 | GCUCCAGACCGGCUGCGAGC | 0.3 |
| 34 | CCGGCUGCGAGCAAAACAAG | 0.3 |
| 35 | UUUGCUCCAGACCGGCUGCG | 0.3 |
| 36 | UGCUCCAGACCGGCUGCGAG | 0.3 |
| 37 | ACCGGCUGCGAGCAAAACAA | 0.3 |
| 38 | GUUGCCGGGCAACGGGGUAA | 30 |
| 39 | UUGCCGGGCAACGGGGUAAA | 30 |
| 40 | GCCGGGCAACGGGGUAAAGG | 30 |
| 41 | CCGGGCAACGGGGUAAAGGU | 3.0 |
| 42 | CGGGCAACGGGGUAAAGGUU | 3.0 |
| 43 | CAGUGGGGGUUGCGUCAGCA | 30 |
| 44 | AGUGGGGGUUGCGUCAGCAA | 30 |
| 45 | GUGGGGGUUGCGUCAGCAAA | 30 |
| 46 | UGGGGGUUGCGUCAGCAAAC | 3.0 |
| 47 | GGGGGUUGCGUCAGCAAACA | 30 |
| 48 | GGGGUUGCGUCAGCAAACAC | 30 |
| 49 | GGUUGCGUCAGCAAACACUU | 0.3 |
| 50 | UCGCCAACUUACAAGGCCUU | 30 |
| 51 | CUCGCCAACUUACAAGGCCU | 3.0 |
| 52 | UUCUCGCCAACUUACAAGGC | 3.0 |
| 53 | UUUCUCGCCAACUUACAAGG | 3.0 |
| 54 | CUUUCUCGCCAACUUACAAG | 30 |
| 55 | ACUUUCUCGCCAACUUACAA | 3.0 |
| 56 | UGAACCUUUACCCCGUUGC | 0.3 |
| 57 | CCUUUACCCCGUUGCCCGGC | 0.3 |
| 58 | GAUCCAUACUGCGGAACUCCU | 0.3 |
| 59 | GCUUGUUUUGCUCGCAGCC | 0.3 |
| 60 | GCUUGUUUUGCUCGCAGCCGG | 30 |
| 61 | UUGUUUUGCUCGCAGCCGG | 30 |
| 62 | UUUUGCUCGCAGCCGGUCU | 3.0 |
| 63 | UUUUGCUCGCAGCCGGUCUGG | 30 |
| 64 | GCUCGCAGCCGGUCUGGAGC | 30 |
| 65 | CUUGUUUUGCUCGCAGCCGG | 0.3 |

Certain oligonucleotides shown to have antiviral activity were selected for modification. In particular, an oligonucleotide of SEQ ID NO: 1 was modified to produce an oligonucleotide of SEQ ID NO: 66. An oligonucleotide of SEQ ID NO: 2 was modified to produce an oligonucleotide of SEQ ID NO: 67. An oligonucleotide of SEQ ID NO: 3 was modified to produce an oligonucleotide of SEQ ID NO: 68. An oligonucleotide of SEQ ID NO: 4 was modified to produce an oligonucleotide of SEQ ID NO: 69. An oligonucleotide of SEQ ID NO: 5 was modified to produce an oligonucleotide of SEQ ID NO: 70. An oligonucleotide of SEQ ID NO: 6 was modified to produce an oligonucleotide of SEQ ID NO: 71. An oligonucleotide of SEQ ID NO: 7 was modified to produce an oligonucleotide of SEQ ID NO: 72. An oligonucleotide of SEQ ID NO: 8 was modified to produce an oligonucleotide of SEQ ID NO: 74. An oligonucleotide of SEQ ID NO: 9 was modified to produce an oligonucleotide of SEQ ID NO: 75. An oligonucleotide of SEQ ID NO: 10 was modified to produce an oligonucleotide of SEQ ID NO: 76. An oligonucleotide of SEQ ID NO: 11 was modified to produce an oligonucleotide of SEQ ID NO: 77. An oligonucleotide of SEQ ID NO: 12 was modified to produce an oligonucleotide of SEQ ID NO: 78. An oligonucleotide of SEQ ID NO: 13 was modified to produce an oligonucleotide of SEQ ID NO: 79. The oligonucleotides were modified according to the following procedure.

Synthesis of 2′-O-Me phosphorothioate oligonucleotides. Each oligonucleotide was synthesized in a Mermade 12 synthesizer (Bioautomation, Plano, Tex.) at the 1 or 2 μmole scale using standard solid supports with a long chain alkylamine controlled-pore derivatized with N⁶-benzoyl-2′-OMe-adenosine, 2′-OMe-uridine, or N⁶-isobutiryl-2′-OMe-guanosine. When the 3′-most nucleotide was 5-methyl-2′-O-methyl-cytosine, universal linker solid support (ChemGenes, MA) was used instead. For 3′-GalNac and 3′-tocopherol conjugated oligonucleotides the corresponding modified solid supports were used. The cycle used followed the steps of: deblocking-coupling-capping-oxidation-capping. 2′-O-methyl phosphoramidites were prepared as 0.1 M solutions in dry acetonitrile. 5-Ethylthiotetrazole was used as activator, 3% trichloroacetic acid in dichloromethane was used to detritylate, acetic anhydride in THF and 16% N-methylimidazole in THF were used to cap, and a 0.1 M solution of xanthane hydride in pyridine was used for sulfurization. Detrytilation was carried out for (2×45) s. Quantitative couplings were achieved in (2×360) s for all bases. Each capping step was carried out for 90 sec. Sulfurization was accomplished in 120 sec. Deprotection and cleavage from the solid support was achieved with ammonia methylamine (AMA) for 15 min at 65° C. When the universal linker was used, the deprotection was left for 1 h at 65° C. After filtering to remove the solid support, the deprotection solution was removed under vacuum in a GeneVac centrifugal evaporator. Crudes were dissolved in 1M PBS and desalted by Ultrafiltration with Vivaspin-Hydrosart-2000 MWCO (Generon Ltd., Berkshire, UK). The purity and molecular weight were determined by HPLC analysis (60° C., IEX-Thermo DNAPac PA-100, A-25 mM sodium phosphate 10% acetonitrile pH 11, B-1.8M NaBr 25 mM sodium phosphate 10% acetonitrile pH 11; RPIP-Waters XBridge OST C18, A-100 mM HFIP 7 mM TEA B-7:3 methanol/acetonitrile) and ESI-MS analysis using Promass Deconvolution for Xcalibur. The purity and molecular weight were determined by HPLC analysis (60° C., IEX-Thermo DNAPac PA-100, A-25 mM sodium phosphate 10% acetonitrile pH 11, B-1.8 M NaBr 25 mM sodium phosphate 10% acetonitrile pH 11; RPIP-Waters XBridge OST C18, A-100 mM HFIP 7 mM TEA B-7:3 methanol/acetonitrile) and ESI-MS analysis using Promass Deconvolution for Xcalibur (Novatia, Newtown, Pa.).

In addition, an oligonucleotide of SEQ ID NO: 7 was modified to produce an oligonucleotide of SEQ ID NO: 73 according to the following procedure.

Synthesis of 3′-amino-2′-deoxy phosphorothioate oligonucleotides. 3′-amino-2′-deoxy phosphorothioate oligonucleotides were synthesized as described in Zielinska, D.; Pongracz, K; Gryaznov, S. M., Tetrahedron Lett. 47, 4495-4499 (2006). 3′-amino-2′-deoxy phosphorothioate phosphoramidite monomers were custom synthesized at Pharmaron. All the monomers were dried in a vacuum desiccator with desiccants (KOH and P₂O₅, at room temperature for 24 hours). Solid supports with a long chain alkylamine controlled-pore attached to the first 5′ residue were obtained from Prime Synthesis (Aston, Pa.). RNA oligonucleotides were synthesized on a Mermade 12 Synthesizer (Bioautomation, Plano, Tex.) using standard oligonucleotide phosphoramidate chemistry starting from the 5′ residue of the oligonucleotide preloaded on solid support using a deblock-coupling-oxidation-coupling-oxidation-capping cycle. Phosphoramidites were prepared as 0.1 M solutions in dry acetonitrile. 5-Ethylthiotetrazole was used as activator, 5% dichloroacetic acid in dichloromethane was used to detritylate (50 sec). Isobutyric anhydride-Lutidine-ACN 1:1:8 and N-methylimidazole in ACN were used to cap, and a 0.1 M solution of xanthane hydride in pyridine was used for sulfurization. Optimal couplings were achieved in (2×120) s for all bases. The capping step was carried out for 90 s. Sulfurization was accomplished in 240 s (split in two couplings). Deprotection and cleavage from the solid support was achieved with aqueous ammonia for 5 h at 55° C. After filtration to remove the solid support, the deprotection solution was removed under vacuum in a Genevac centrifugal evaporator. Crude product was then purified on GE Akta Explorer using ion exchange chromatography (Source 15Q, 20 mM NaH₂PO₄, 15% CH₃CN, 1.8 M NaBr, gradient 5-50% B over 20 column volumes) and fractions were analyzed by reverse phase ion pair chromatography on a Thermo HPLC. Pure fractions were pooled and desalted by ultrafiltration (using Vivaspin-Hydrosart-2000 MWCO (Generon Ltd., Berkshire, UK)) and were evaporated to dryness on a GeneVac Evaporator. The purity and molecular weight were determined by HPLC analysis (60° C., IEX-Thermo DNAPac PA-100, A-25 mM sodium phosphate 10% acetonitrile pH 11, B-1.8 M NaBr 25 mM sodium phosphate 10% acetonitrile pH 11; RPIP-Waters XBridge OST C18, A-100 mM HFIP 7 mM TEA B-7:3 methanol/acetonitrile) and ESI-MS analysis using Promass Deconvolution for Xcalibur (Novatia, Newtown, Pa.). The purified oligonucleotides were analyzed as described above for the synthesis of 2′-O-Me phosphorothioate oligonucleotides.

Table 4 provides a summary of oligonucleotides of the present disclosure chosen for further modification, their nucleotide length, the sequences that are targeted by the oligonucleotides and the direction of the oligonucleotide.

TABLE 4

| | | HBV Genome Start | HBV Genome Stop | |
|---|---|---|---|---|
| | | (HBV DNA | (HBV DNA | Direction of Oligo: |
| | | Nucleotide Index | Nucleotide Index | Antisense (AS) or |
| Oligonucleotide | Length | From 5′ to 3′) | From 5′ to 3′) | Sense (SS) |
| SEQ ID NO: 66 | 21 | 1194 | 1214 | AS |
| SEQ ID NO: 67 | 19 | 1198 | 1216 | AS |
| SEQ ID NO: 68 | 20 | 1094 | 1113 | AS |
| SEQ ID NO: 69 | 19 | 969 | 987 | AS |
| SEQ ID NO: 70 | 17 | 986 | 970 | SS |
| SEQ ID NO: 71 | 19 | 987 | 969 | SS |
| SEQ ID NO: 72 | 20 | 1155 | 1136 | SS |
| SEQ ID NO: 73 | 20 | 1155 | 1136 | SS |
| SEQ ID NO: 74 | 20 | 1175 | 1194 | AS |
| SEQ ID NO: 75 | 20 | 1174 | 1193 | AS |
| SEQ ID NO: 76 | 20 | 1116 | 1097 | SS |
| SEQ ID NO: 77 | 14 | 986 | 973 | SS |
| SEQ ID NO: 78 | 19 | 1193 | 1175 | SS |
| SEQ ID NO: 79 | 19 | 1315 | 1297 | SS |

The EC50 and CC50 values for each oligonucleotide are summarized in Table 5. As shown in Table 5, the oligonucleotides of the present disclosure were effective in inhibiting the expression levels of HBV viral antigens (HBeAg and HBsAg) at low concentrations. Indeed, the EC50 values for the oligonucleotides ranged from 0.013 μM to 0.35 μM for HBeAg inhibition, and 0.018 μM to 1.12 μM for HBsAg inhibition. See Table 5.

TABLE 5

| | HBeAg | HBsAg | CC50 |
|---|---|---|---|
| Oligonucleotide | EC50 (μM) | EC50 (μM) | (μM) |
| SEQ ID NO: 66 | 0.35 | 0.02 | >30 |
| SEQ ID NO: 67 | | 0.32 | >30 |
| SEQ ID NO: 68 | 0.19 | 1.12 | >10 |
| SEQ ID NO: 69 | 0.78 | 0.81 | >10 |
| SEQ ID NO: 70 | 0.04 | 0.09 | 6.6 |
| SEQ ID NO: 71 | 0.020/0.036 | 0.024/0.04 | >10 |
| SEQ ID NO: 72 | 0.036/0.19 | 0.09/0.32 | >10 |
| SEQ ID NO: 73 | 0.13 | 1.1 | 3.3 |
| SEQ ID NO: 74 | 0.29 | 0.34 | >10 |
| SEQ ID NO: 75 | 0.43 | 0.21 | >10 |
| SEQ ID NO: 76 | 0.025 | 0.064 | 1.37 |
| SEQ ID NO: 77 | 0.1 | 0.1 | >10 |
| SEQ ID NO: 78 | 0.13 | 0.13 | >1 |
| SEQ ID NO: 79 | 0.013 | 0.018 | >10 |

These results demonstrate that the oligonucleotides of the present disclosure can reduce viral antigens in HBV infected hepatocytes. Accordingly, the oligonucleotides of the present disclosure are useful in methods for treating HBV infection in a subject.

SEQ ID NOs: 70-72, 74 and 75 were also tested for reduction in HBV DNA as set forth in Table 6.

TABLE 6

| | EC₅₀ sup HBV DNA |
|---|---|
| Oligo | (μM) |
| SEQ ID NO: 70 | 0.051 |
| SEQ ID NO: 71 | 0.11 |
| SEQ ID NO: 72 | 0.46 |
| SEQ ID NO: 74 | 0.23 |
| SEQ ID NO: 75 | 0.18 |

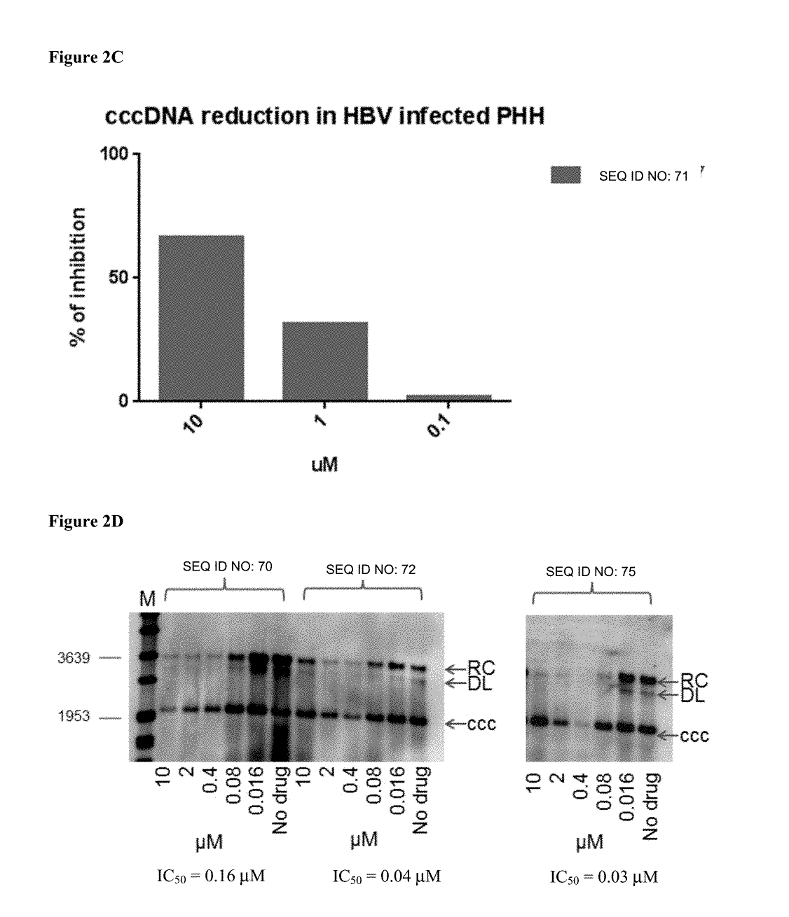

Further, FIG. 2A demonstrates that SEQ ID NO: 71 decreased rcDNA and cccDNA levels in HBV infected PHH at concentrations ranging between 10 μM and 0.08 μM compared to untreated controls in a dose dependent manner. FIGS. 2B-2C demonstrate that treatment with SEQ ID NO: 71 resulted in a reduction in cccDNA levels compared to untreated controls in a dose dependent manner (e.g., 66.3% reduction at 10 μM).

FIG. 2D demonstrates that treatment with SEQ ID NO: 70, SEQ ID NO: 72 and SEQ ID NO: 75 resulted in a dose dependent reduction of cccDNA levels in HBV infected PHH (about 50-75% maximal reduction of cccDNA levels). The IC₅₀ values of SEQ ID NO: 70, SEQ ID NO: 72 and SEQ ID NO: 75 were 0.16 μM, 0.04 μM, and 0.03 μM, respectively.

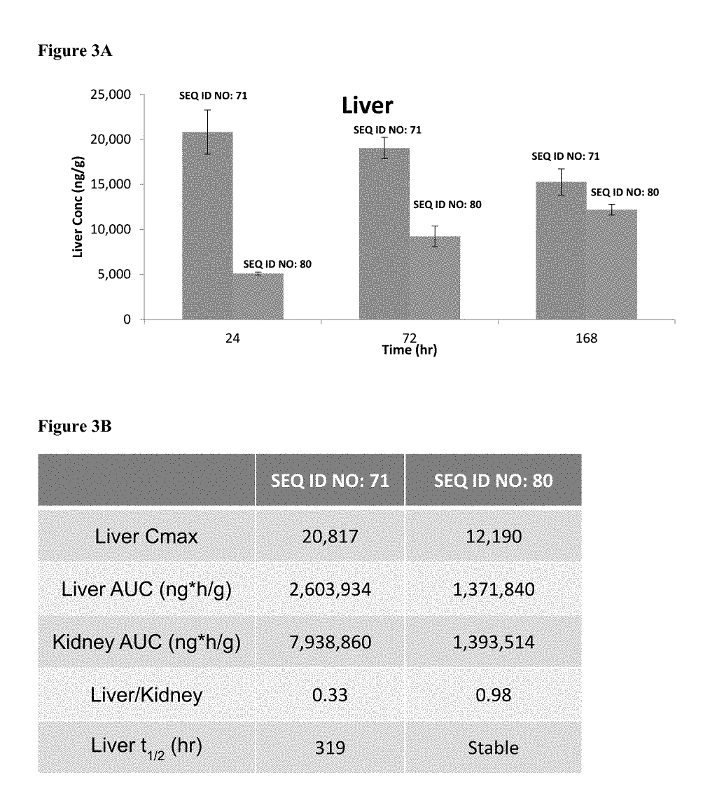

FIGS. 3A-3B show the in vivo liver concentrations and liver half-lives for SEQ ID NO: 71 and SEQ ID NO: 80 in C57B1/6 female mice. The liver half-life for SEQ ID NO: 71 was 319 hours. The liver half-life for SEQ ID NO: 80 was stable. Similar patterns were observed with other cccDNA targeting oligonucleotides that are disclosed herein. These results demonstrate that the cccDNA targeting oligonucleotides of the present disclosure are delivered to the liver in vivo.

Immunocompromised FRG mice were infected with HBV and allowed to reach stable viremia before sacrifice. The infected hepatocytes were plated and treated immediately with 3 different concentrations of the indicated oligonucleotides (in triplicate). HBV cccDNA levels were assessed by Southern Blot 9 days after treatment. Treatment with SEQ ID NO: 80 resulted in >60% reduction in cccDNA levels in ex vivo HBV infected FRG (Fah^{-/-}/Rag2^{-/-}/Il2rg^{-/-}) mouse hepatocytes compared to untreated controls.

Ex vivo PXB hepatocytes were infected with HBV and treated with different concentrations of the indicated oligonucleotides (free uptake) at day −1 (1 day prior to infection), day 0 (same day as infection), and day 1 (1 day after infection). cccDNA levels were assessed by qPCR at day 12 post infection. Treatment with SEQ ID NO: 82 resulted in >50% reduction in cccDNA levels in ex vivo PXB hepatocytes that were infected with HBV. Table 7 presents a summary of HBsAg EC₅₀, cccDNA reduction, and liver AUC for SEQ ID NOs: 71, 80 and 82.

TABLE 7

| | | HBsAg | | |
|---|---|---|---|---|
| SEQ | | EC₅₀ (μM) - | cccDNA | Liver AUC |
| ID NO: | Modification | PHH, transfection | reduction | (ng * h/g) |
| 71 | — | 0.024 | >60% (PHH) | 2,603,934 |
| 80 | 3′GalNAc | 0.024* | >60% (FRG) | 1,371,840 |
| 82 | 3′Toco | | >50% (PXB) | |

*EC₅₀ value for unconjugated parent oligo (SEQ ID NO: 71)

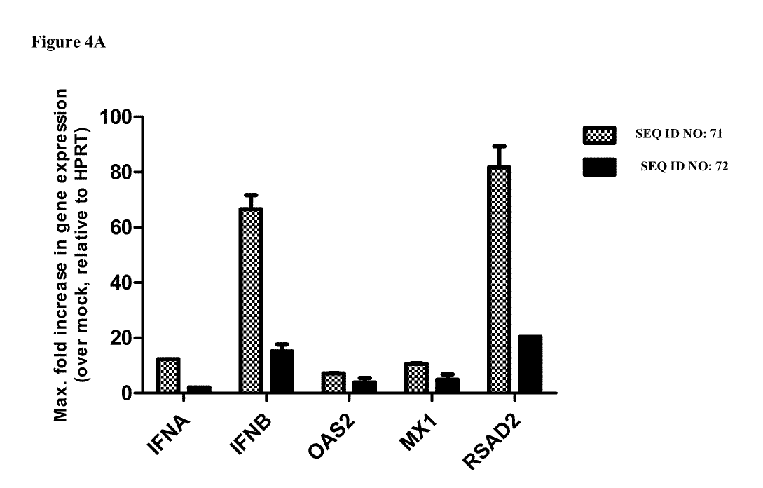

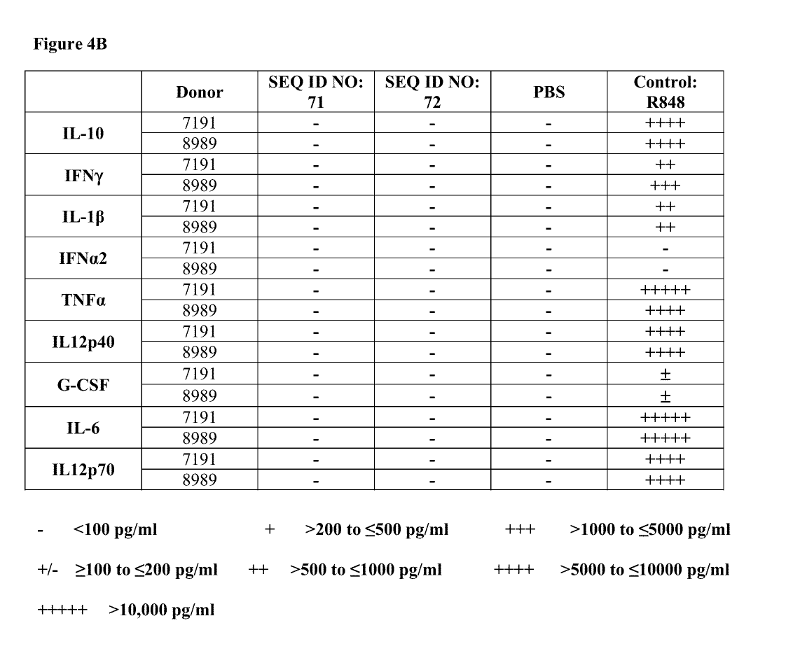

FIG. 4A shows that IFN-stimulated genes (ISGs) were upregulated in HBV-infected PHHs upon treatment with SEQ ID NO: 71 and SEQ ID NO: 72, which is consistent with the formation of D-loop structures within these treated cells.

FIG. 4B demonstrate that cytokines were not induced in HBV negative cells that were contacted with SEQ ID NO: 71 and SEQ ID NO: 72, thus demonstrating that the immune response observed in FIG. 4A was specific for cccDNA in HBV-infected cells.

These results demonstrate that the oligonucleotides of the present disclosure can reduce viral antigens and viral DNA in HBV infected hepatocytes in a selective manner. Accordingly, the oligonucleotides of the present disclosure are useful in methods for treating HBV infection in a subject.

### Equivalents

The present disclosure is not to be limited in terms of the particular embodiments described in this application, which are intended as single illustrations of individual aspects of the present disclosure. Many modifications and variations of this present disclosure can be made without departing from its spirit and scope, as will be apparent to those skilled in the art. Functionally equivalent methods and apparatuses within the scope of the present disclosure, in addition to those enumerated herein, will be apparent to those skilled in the art from the foregoing descriptions. Such modifications and variations are intended to fall within the scope of the present disclosure. It is to be understood that this present disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. An oligonucleotide comprising a sequence that is complementary to a plurality of nucleotides within an HBV cccDNA genome sequence of SEQ ID NO: 100.

2. The oligonucleotide of claim 1, wherein the oligonucleotide is complementary to at least 12 nucleotides within the Enhancer I region of the HBV cccDNA genome.

3. An oligonucleotide comprising a sequence that is complementary to at least 12 nucleotides that are present in a genome region corresponding to nucleotide position 967 to nucleotide position 1322 of an HBV cccDNA genome.

4. The oligonucleotide of claim 3, wherein the sequence of the oligonucleotide is selected from the group consisting of SEQ ID NOs: 1-65.

5. The oligonucleotide of claim 1, wherein the oligonucleotide contains at least one first nucleotide having a phosphorothioate (PS) linkage to a second nucleotide, wherein said first nucleotide is modified at the 2′ position with a substitution selected from the group consisting of F and O-alkyl, wherein said O-alkyl is optionally substituted with alkoxy.

6. The oligonucleotide of claim 5, wherein each nucleotide of the oligonucleotide having a cytosine nucleobase is modified to be a 2′-O-Me, 5-methylcytosine (5mmC); each other nucleotide is modified to include an O-Me modification at the 2′ position (mA, mG and mU); and each nucleotide contains a phosphorothioate (PS) linkage between nucleotides.

7. The oligonucleotide of claim 6, wherein the sequence of the oligonucleotide is selected from the group consisting of SEQ ID NOs: 66-72 and 74-82.

8. The oligonucleotide of claim 1, wherein the oligonucleotide contains at least one first nucleotide having a thiophosphoroamidate (NPS) linkage to a second nucleotide, wherein said first nucleotide is modified at the 2′ position with a substitution selected from the group consisting of F and O-alkyl, wherein said O-alkyl is optionally substituted with alkoxy.

9. The oligonucleotide of claim 8, wherein each nucleotide of the oligonucleotide having a cytosine nucleobase is modified to include a 2′-O-Me, 5-methylcytosine (5mmC); each other nucleotide is modified to include an O-Me modification at the 2′ position (mA, mG and mU); and each nucleotide contains a thiophosphoroamidate (NPS) linkage between nucleotides.

10. The oligonucleotide of claim 9, wherein the sequence of the oligonucleotide is SEQ ID NO: 73.

11. The oligonucleotide of claim 1, further comprising at least one targeting moiety conjugated to the oligonucleotide.

12. The oligonucleotide of claim 11, wherein the targeting moiety conjugated to the oligonucleotide is selected from the group consisting of GalNAc, palmitoyl and tocopherol derivatives.

13. The oligonucleotide of claim 12, wherein the sequence of the oligonucleotide is selected from the group consisting of SEQ ID NOs: 80-82.

14. A pharmaceutical composition comprising at least one oligonucleotide of claim 1.

15. A method of treating HBV in a subject in need thereof comprising administering to the subject an effective amount of the oligonucleotide of claim 1.

16. The method of claim 15, wherein the sequence of the oligonucleotide is selected from the group consisting of SEQ ID NOs: 1-82.

17. The method of claim 15, wherein administration of the oligonucleotide results in a decrease in at least one of HBeAg levels, HBsAg levels or HBV DNA levels in the subject.

18. The method of claim 15, wherein administration of the oligonucleotide results in a reduction of HBV cccDNA in the subject.

19. The method of claim 15, further comprising separately, sequentially or simultaneously administering to the subject one or more additional therapeutic agents selected from the group consisting of: an antiviral agent, a nucleotide analog, a nucleoside analog, a reverse transcriptase inhibitor, an immune modulator, a therapeutic vaccine, a viral entry inhibitor, a capsid inhibitor, a siRNA, an antisense oligonucleotide, and a cccDNA inhibitor.

20. An oligonucleotide of claim 1 for use in the treatment of HBV.
